(19) United States
(12) Patent Application Publication
(10) Pub. No.: US 2009/0041778 A1
(43) Pub. Date: Feb. 12, 2009

(54) Methods And Compositions For The Treatment Of Graft Failure

(76) Inventor: Vikas P. Sukhatme, Newton, MA (US)

Correspondence Address: CLARK & ELBING LLP, 101 FEDERAL STREET, BOSTON, MA, 02110, US

(21) Appl. No.: 11/791,293

(22) PCT Filed: Nov. 21, 2005
(86) PCT No.: PCT/US05/42111

§ 371 Date: Feb. 27, 2008

Related U.S. Application Data
(60) Provisional application No. 60/630,242, filed on Nov. 22, 2004.

(51) Int. Cl. A61K 39/395; A61K 31/436

(52) U.S. Cl. 424/158.1; 424/172.1; 514/291

(57) ABSTRACT

The present invention provides methods and compositions for treating graft failure resulting from neointimal hyperplasia. These methods and compositions feature the use of platelet derived growth factor receptor (PDGFR) inhibitor compounds, such as N-phenyl-2-pyrimidine compounds (e.g., imatinib mesylate), or mTOR inhibitors (e.g., rapamycin).

### BACKGROUND OF THE INVENTION

The invention provides methods and compositions for treating graft failure.

Patients suffering from kidney failure, such as diabetics and patients suffering from end stage renal disease, undergo hemodialysis to remove waste products such as urea from the blood. Hemodialysis requires vascular access. The most common access method is via a synthetic graft made from polytetrafluoroethylene (PTFE). One end of the graft is connected to a vein and the other end is connected to an artery, thus creating a site to connect the hemodialysis needles. Unfortunately, the PTFE graft is subject to graft thrombosis. Vascular access dysfunction is responsible for approximately 20% of all hospitalizations in the end-stage renal disease population at a cost of $1 billion per annum. In the majority of these cases, the underlying pathology is neointimal hyperplasia and stenosis at the venous anastamotic site or in the downstream vein.

Neointimal hyperplasia is also associated with grafts used to treat peripheral vascular disease. PVD affects approximately 8 million people in the United States alone. In peripheral vascular disease the arteries that carry blood to the arms or legs become narrowed or clogged. This narrowing, which is most often due to atherosclerosis, is a gradual process in which cholesterol and scar tissue build up, forming a “plaque” that clogs the blood vessel. This condition can be treated using a by-pass graft to redirect the circulation of blood around the site of the arterial block. Typically, a synthetic graft or a vein graft from another part of the body is used to create a detour around the blocked artery. Alternatively, a vein interposition graft can be used in which a segment of vein is used to replace the occluded section of artery. Unfortunately, by-pass grafts used to treat PVD are also subject to hyperplasia, which can ultimately lead to graft failure.

There are no effective therapeutic treatments known to prevent or treat neointimal hyperplasia leading to graft failure associated with vascular access dysfunction or PVD. Clearly, effective therapeutics aimed at the prevention of graft failure in the treatment of these diseases are required.

### SUMMARY OF THE INVENTION

The present invention provides methods and compositions for treating graft failure resulting from neointimal hyperplasia. These methods and compositions feature the use of platelet derived growth factor receptor (PDGFR) inhibitor compounds, such as N-phenyl-2-pyrimidine compounds (e.g., imatinib mesylate) to inhibit the biological activity of the PDGFR. The invention also features methods of treating or preventing graft failure resulting from neointimal hyperplasia in a patient by administering to the patient an mTOR inhibitor (e.g., rapamycin).

In a first aspect, the invention generally features a method for the prevention or treatment of graft failure resulting from neointimal hyperplasia, the method involves administering to a patient an effective amount of an N-phenyl-2-pyrimidine compound of the formula:

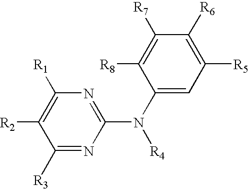

where R₁ is hydrogen or C₁-C₃ alkyl; R₂ is hydrogen or C₁-C₃ alkyl, R₃ is 2-pyridyl, 3-pyridyl, 4-pyridyl, 2-methyl-3-pyridyl, 4-methyl-3-pyridyl, 2-furyl, 5-methyl-2-furyl, 2,5-dimethyl-3-furyl, 2-thienyl, 3-thienyl, 5-methyl-2-thienyl, 2-phenothiazinyl, 4-pyrazinyl, 2-benzofuryl, N-oxido-2-pyridyl, N-oxido-3-pyridyl, N-oxido-4-pyridyl, 1H-indol-2-yl, 1H-indol-3-yl, 1-methyl-1H-pyrrol-2-yl, 4-quinolinyl, 1-methyl-pyridinium-4-yliodide, dimethylaminophenyl, or N-acetyl-N-methyl-aminophenyl; R₄ is hydrogen, C₁-C₃ alkyl, —CO—CO—O—C₂H₅, or N,N-dimethylaminoethyl; at least one of R₅, R₆, R₇, and R₈ is C₁-C₆ alkyl, C₁-C₃ alkoxy, chloro, bromo, iodo, trifluoromethyl, hydroxy, phenyl, amino, mono(C₁-C₃-alkyl)amino, di(C₁-C₃ alkyl)amino, C₂-C₄ alkanoyl, propenyloxy, carboxy, carboxymethoxy, ethoxycarbonylmethoxy, sulfanilamido, N,N-di(C₁-C₃ alkyl)sulfanilamido, N-methylpiperazinyl, piperidinyl, 1H-imidazol-1-yl, 1H-triazol-1-yl, 1H-benzimidazol-2-yl, 1-naphthyl, cyclopentyl, 3,4-dimethylbenzyl, or a radical of one of the formulae:

—CO₂R, —NHC(═O)R, —N(R)CH₂C(═O)R, —O(CH₂)ₙN(R)R, —CH₂(C═O)NH(CH₂)ₙN(R)R, —CH(CH₃)NHCHO, —CH(CH₃)C═N—OH, —CH(CH₃)C═N—O—CH₃, —CH(CH₃)NH₂, —NHCH₂CH₂(C═O)N(R)R,

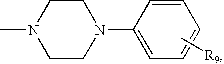

—(CH₂)ₘR₁₀, —X—(CH₂)ₘR₁₀, or

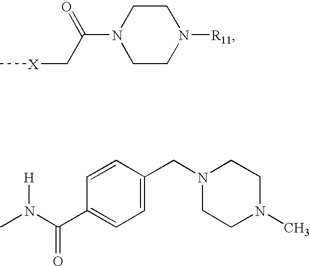

where R is C₁-C₃ alkyl, X is oxygen, or sulfur, m is 1, 2, or 3, n is 2 or 3; R₉ is hydrogen, C₁-C₃ alkyl, C₁-C₃ alkoxy, chloro, bromo, iodo, or trifluoromethyl; R₁₀ is 1H-imidazol-1-yl or morpholinyl; and R₁₁ is C₁-C₃ alkyl or unsubstituted phenyl, or phenyl that is monosubstituted by C₁-C₃ alkyl, halogen or trifluoromethyl; and the remaining of the substituents R₅, R₆, R₇, and R₈ are hydrogen; or a pharmaceutically acceptable salt of the N-phenyl-2-pyrimidine compound containing at least one salt-forming group where the compound inhibits PDGFR biological activity and the administering is in a dose sufficient to prevent or treat the graft failure.

The compounds of Formula I and their preparation are disclosed in EP-A-0 233 461 and in U.S. Pat. Nos. 4,876,252; 5,516,775; 5,705,502; and 5,521,184, incorporated herein by reference.

In one preferred embodiment, the compound is of the formula

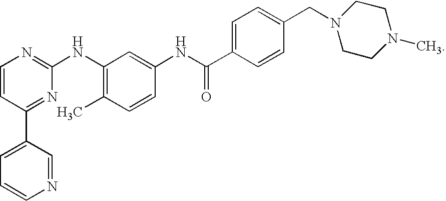

This compound is hereafter referred to as imatinib mesylate (also known as Gleevec™).

In another aspect, the invention features a method for the prevention or treatment of graft failure resulting from neointimal hyperplasia by administering to a patient an effective amount of an mTOR inhibitor (e.g., rapamycin), wherein the administering is in a dose sufficient to prevent or treat the graft failure.

In preferred embodiments of either of the foregoing aspects, the graft is used for vascular access in hemodialysis or is used to treat peripheral vascular disease. In various embodiments, the graft failure is characterized by the migration of smooth muscle cells into the intima, the proliferation of vascular smooth muscle cells, or the deposition of extracellular matrix. In another embodiment, graft failure is the result of vascular stenosis or thrombosis. In other embodiments, vascular access for hemodialysis is associated with the use of a polytetrafluoroethylene (PTFE) graft or is associated with the use of an arteriovenous fistula. In another embodiment, the graft is comprised of a synthetic material (e.g., Dacron). In one preferred embodiment, the graft contains a portion of vein from the patient who will receive the graft. In another embodiment, the compound is given in combination with a pharmaceutically acceptable carrier. In another embodiment, the dosage is sufficient to prevent or ameliorate vascular stenosis or thrombosis. In one preferred embodiment, the dosage is sufficient to prevent or ameliorate neointimal hypelplasia.

In another aspect, the invention features a pharmaceutical composition containing (i) a compound of formula (I) or an mTOR inhibitor and (ii) one or more additional compounds selected from the group consisting of: an angiogenesis inhibitor, an anti-proliferative compound, an immunosuppressive compound, an anti-migratory compound, an anti-platelet agent, and an anti-fibrotic compound.

In preferred embodiments of the first aspect, the method further involves administering to a patient at least one additional compound selected from the group consisting of, an angiogenesis inhibitor, an anti-proliferative compound, an immunosuppressive compound, an anti-migratory compound, an anti-platelet compound, and an anti-fibrotic compound.

In another aspect, the invention features a kit containing (i) an N-phenyl-2-pyrimidine compound, (ii) a second compound that is selected from the group consisting of: an angiogenesis inhibitor, an anti-proliferative compound, an immunosuppressive compound, an anti-migratory compound, an anti-platelet compound, and an anti-fibrotic compound; and (iii) instructions for administering the N-phenyl-2-pyrimidine compound and the second compound to a patient diagnosed with or at risk of developing graft failure resulting from neointimal hyperplasia. In one preferred embodiment, the N-phenyl-2-pyrimidine compound is imatinib mesylate. In other preferred embodiments, the graft is used for vascular access in hemodialysis or to treat peripheral vascular disease. In another embodiment, graft failure is characterized by the migration of smooth muscle cells into the intima, by the proliferation of vascular smooth muscle cells, or by the deposition of extracellular matrix. In another embodiment, the graft failure is the result of vascular stenosis or thrombosis. In preferred embodiments, the second compound is selected from the group consisting of: an angiogenesis inhibitor, an anti-proliferative compound, an immunosuppressive compound, an anti-migratory compound, an anti-platelet compound, and an anti-fibrotic compound.

In another aspect, the invention features a method for identifying combinations of compounds useful for treating graft failure caused by neointimal hyperplasia in a patient in need of such treatment, the method involves the steps of: (a) contacting human aortic vascular smooth muscle cells in vitro with (i) an N-phenyl-2-pyrimidine compound and (ii) a candidate compound; and (b) determining whether the combination of the N-phenyl-2-pyrimidine compound and the candidate compound reduces human aortic vascular smooth muscle cells migration to a greater extent than when cells are contacted with an N-phenyl-2-pyrimidine compound alone, where a reduction of the cell migration identifies the combination as a combination that is useful for treating or preventing graft failure caused by neointimal hyperplasia in a patient in need of such treatment.

In another aspect, the invention features a delivery formulation containing an N-phenyl-2-pyrimidine compound dispersed in a polymer. In another preferred embodiment, the delivery formulation further contains a second compound selected from the group consisting of: an angiogenesis inhibitor, an anti-proliferative compound, an immunosuppressive compound, an anti-migratory compound, an anti-platelet compound, and an anti-fibrotic compound. In another preferred embodiment, the polymer is in the form of a microsphere. In another preferred embodiment, the polymer is in the form of a film. In another preferred embodiment, the polymer is in the form of a suture. In another preferred embodiment, the polymer is in the form of a composite system that includes a microsphere embedded in a hydrophobic matrix.

In preferred embodiments of the previous aspects, an angiogenesis inhibitor is any one or more of the following an antibody, an antibody that binds VEGF-A, an antibody binds a VEGF receptor and blocks VegF binding, avastin, endostatin, angiostatin, restin, tumstatin, TNP-470, 2-methoxyestradiol, thalidomide, a peptide fragment of an anti-angiogenic protein, canstatin, arrestin, a VEGF kinase inhibitor, VEGF kinase inhibitor, CPTK787, SFH-1, an anti-angiogenic protein, thrombospondin-1, platelet factor-4, interferon-α, an agent that blocks TIE-1 or TIE-2 signalling, PIH12 signalling, an agent that blocks an extracellular vascular endothelial (VE) cadherin domain, an antibody that binds to an extracellular VE-cadherin domain, tetracycline, penicillamine, vinblastine, cytoxan, edelfosine, tegafur or uracil, curcumin, green tea, genistein, resveratrol, N-acetyl cysteine, captopril, a cox-2 inhibitor, celecoxib (CELEBREX), and rofecoxib (VIOXX).

In other preferred embodiments of the previous aspects, an anti-proliferative compound is any one or more of the following: rapamycin, everolimus, taxol, troglitazone, an antibody that binds bFGF, an antibody that binds bFGF-saporin, a statin, an ACE inhibitor, suramin, 17 beta-estradiol, atorvastatin, fluvastatin, lovastatin, pravastatin, simvastatin, cerivastatin, perindopril, quinapril, captopril, lisinopril, enalapril, fosinopril, cilazapril, ramipril, a kinase inhibitor.

In yet other preferred embodiments of the previous aspects, an immunosuppressive compound is any one or more of the following: prednisone, FTY720, methylprednisolone, α-tocopherol, azathioprine, chlorambucil, cyclophosphamide, an antibody that binds to an IL-2 receptor or to CTLA4, methotrexate, mycophenolate mofetil, cyclosporine, an agent that interferes with macrophage function, an agent that inhibits P-selectin biological function, an agent that PSGL-1 biological function, an agent that VLA-4 biological function, an agent that VCAM-1 biological function, is an agent that Mac-1 biological function, and FTY720.

In other preferred embodiments of the previous aspects, an anti-migratory compound is any one or more of the following: cyproheptadine, methysergide, bosentan, YM087, cyproheptadine, ketanserin, and anplag.

In other preferred embodiments, an anti-platelet compound is any one or more of the following: ticlopidine, cilostazol, dipyridamole, abciximab, clopidogrel, , a glycoprotein iib/iiia inhibitor, eptifibatide, tirofiban, and a phosphodiesterase III inhibitor.

In another preferred embodiment, an anti-fibrotic compound is any one or more of the following: an agent that blocks TGF-β signaling or inhibits activation of plasminogen activator inhibitor-1 promoter activity, an antibody that binds to TGF-β or to a TGF-β receptor, an antibody that binds to a TGF-β receptor I, II, or III, a kinase inhibitor, an agent that blocks connective tissue growth factor (CTGF) signaling, an agent that inhibits prolyl hydroxylase, an agent that inhibits procollagen C-proteinase, pirfenidone, silymarin, pentoxifylline, colchicines, embrel, remicade, an agent that antagonizes transforming growth factor beta (TGF-β), an agent that antagonizes connective tissue growth factor (CTGF), and an agent that inhibits vascular endothelial growth factor (VEGF), and an agent that antagonizes connective tissue growth factor (CTGF).

It should be noted that many compounds possess more than one activity. For example, a serotonin receptor antagonist can have both anti-migratory and anti-proliferative activity. These categories are not meant to limit the compounds, but to provide broad descriptions of the potential activities of each compound.

In other preferred embodiments of the previous aspects, an N-phenyl-2-pyrimidine derivative (e.g., imatinib mesylate), includes any N-phenyl-2-pyrimidine compounds, exemplary compounds are described in U.S. Pat. Nos. 4,876,252; 5,516,775; 5,705,502; and 5,521,184, incorporated herein by reference, or any other PDGFR inhibitors.

As used herein, the terms “alkyl” and the prefix “alk-” are inclusive of both straight chain and branched chain saturated or unsaturated groups, and of cyclic groups, i.e., cycloalkyl and cycloalkenyl groups. Unless otherwise specified, acyclic alkyl groups are from 1 to 6 carbons. Cyclic groups can be monocyclic or polycyclic and preferably have from 3 to 8 ring carbon atoms. Exemplary cyclic groups include cyclopropyl, cyclopentyl, cyclohexyl, and adamantyl groups.

By “aryl” is meant a carbocyclic aromatic ring or ring system. Unless otherwise specified, aryl groups are from 6 to 18 carbons. Examples of aryl groups include phenyl, naphthyl, biphenyl, fluorenyl, and indenyl groups.

By “heteroaryl” is meant an aromatic ring or ring system that contains at least one ring hetero-atom (e.g., O, S, or N). Unless otherwise specified, heteroaryl groups are from 1 to 9 carbons. Heteroaryl groups include furanyl, thienyl, pyrrolyl, imidazolyl, pyrazolyl, oxazolyl, isoxazolyl, thiazolyl, isothiazolyl, triazolyl, oxadiazolyl, oxatriazolyl, pylidyl, pyridazyl, pyrimidyl, pyrazyl, triazyl, benzofuranyl, isobenzofuranyl, benzothienyl, indole, indazolyl, indolizinyl, benzisoxazolyl, quinolinyl, isoquinolinyl, cinnolinyl, quinazolinyl, naphtyridinyl, phthalazinyl, phenanthrolinyl, purinyl, and carbazolyl groups.

By “heterocycle” is meant a non-aromatic ring or ring system that contains at least one ring heteroatom (e.g., O, S, or N). Unless otherwise specified, heterocyclic groups are from 1 to 9 carbons. Heterocyclic groups include, for example, dihydropyrrolyl, tetrahydropyrrolyl, piperazinyl, pyranyl, dihydropyranyl, tetrahydropyranyl, tetrahydrofuranyl, dihydrothiophene, tetrahydrothiophene, and morpholinyl groups.

By “halide” or “halogen” or “halo” is meant bromine, chlorine, iodine, or fluorine.

Aryl, heteroaryl, or heterocyclic groups may be unsubstituted or substituted by one or more substituents selected from the group consisting of C₁₋₆ alkyl, hydroxy, halo, nitro, C₁₋₆ alkoxy, C₁₋₆ alkylthio, trifluoromethyl, C₁₋₆ acyl, arylcarbonyl, heteroarylcarbonyl, nitrile, C₁₋₆ alkoxycarbonyl, arylalkyl (wherein the alkyl group has from 1 to 6 carbon atoms), and heteroarylalkyl (wherein the alkyl group has from 1 to 6 carbon atoms).

By “anti-fibrotic agent” is meant any agent, that reduces or inhibits the production of extracellular matrix components including, but not limited to, fibronectin, proteoglycan, collagen, and elastin. Examples of anti-fibrotic agents include, but are not limited to, antagonists of TGFP and CTGF, also agents that block TGF-β signaling or inhibit activation of plasminogen activator inhibitor-1 promoter activity, an antibody that binds to TGF-β or to a TGF-β receptor (e.g., TGF-β receptor I, II, or III), a kinase inhibitor, an agent that blocks connective tissue growth factor (CTGF) signaling, an agent that inhibits prolyl hydroxylase, an agent that inhibits procollagen C-proteinase, pirfenidone, silymarin, pentoxifylline, colchicines, embrel, remicade, an agent that antagonizes transforming growth factor beta (TGF-β)., an agent that antagonizes connective tissue growth factor (CTGF), and an agent that inhibit vascular endothelial growth factor (VEGF)., an agent that antagonizes connective tissue growth factor (CTGF).

By “anti-migratory compound” is meant any compound that blocks the movement or migration of smooth muscle cells. Anti-migratory compounds also include any compound that can inhibit any of the cellular signaling proteins known to induce migration of smooth muscle cells. Examples of compounds with anti-migratory activity include, but are not limited to, serotonin receptor antagonists (e.g., cyproheptadine or methysergide), compounds that antagonize either of the endothelin receptors, ET_{A} and ET_{B}, (e.g., bosentan), and vasopressin receptor antagonists (e.g., YM087). Other anti-migratory compounds include, but are not limited to methysergide, bosentan, YM087, cyproheptadine, ketanserin, and anplag.

By “anti-platelet agent” is meant any compound that can inhibit one or more of the steps leading to platelet activation (i.e., platelet shape change, secretion of platelet granule contents, and aggregation of platelets). Preferably these compounds will inhibit the production of PDGF. Examples of anti-platelet agents include, but are not limited to ticlopidine, cilostazol, abciximab, clopidogrel, dipyridimole, a glycoprotein iib/iiia inhibitor, eptifibatide, tirofiban, and a phosphodiesterase III inhibitor.

By “anti-proliferative compound” is meant any compound that can reduce or inhibit the proliferation of vascular smooth muscle cells or endothelial cells. Anti-proliferative compounds include any compound that can inhibit any of the cellular signaling proteins known to induce proliferation of smooth muscle cells. Examples of mitogens known to induce proliferation of vascular smooth muscle cells include PDGF, bFGF, endothelin-1, angiotensin II, EGF, IGF-1, vasopressin, thrombin, serotonin, and numerous lipid mediators. Also included are of anti-proliferative compounds that can inhibit the mitogenic effects of any of these proteins. Other examples of anti-proliferative compounds include, but are not limited to, rapamycin, everolimus, CCI-779, taxol, troglitazone, an antibody that binds bFGF, an antibody that binds bFGF-saporin, a statin, an ACE inhibitor, suramin, 17 beta-estradiol, atorvastatin, fluvastatin, lovastatin, pravastatin, simvastatin, celivastatin, pelindopril, quinapril, captoplil, lisinopril, enalapril, fosinopril, cilazapril, ramipril, and kinase inhibitors.

By “hemodialysis” is meant a procedure used to remove toxic wastes from the blood of a patient with acute or chronic renal failure. The removal of such waste elements occurs by virtue of differences in rates of their diffusion through a semipermeable membrane while the blood is being circulated outside the body.

By “graft” is meant any type of reconnection used to by-pass or replace an area of a blood vessel that is blocked or occluded. Typically grafts are inserted through procedures where portions of artery or vein from one area of the patient's body are used to reroute blood around a blockage in another area. Non-limiting examples of grafts using the patient's own blood vessels include the use of the patient's long saphenous vein, internal mammary artery, or a Dacron coated umbilical vein. Synthetic grafts can also be used to reroute blood around a blocked artery. Non-limiting examples of synthetic materials used for grafts include PTFE, Dacron, or any type of woven material commonly used for grafts. As used herein, “graft” can also refer to an arteriovenous fistula, which is established surgically by attaching a patient's artery to a vein, or a vein interposition graft in which a section of the patient's vein is actually used to replace the area of vessel that is blocked or occluded.

By “graft failure” is meant any reduction or loss of blood flow over the graft site as a result of neointimal hyperplasia.

By “immunosuppressive compound” is meant any agent that can reduce or inhibit the natural immune response induced by chemical, biological, or physical agents. Preferably, an immunosuppressive compound can inhibit monocyte or macrophage activity in any one of four ways: 1) by decreasing the production or increasing the removal of monocytes, 2) by preventing differentiation of monocytes into macrophages, 3) by preventing monocytes from migrating into the region of hyperplasia, and 4) by blocking the activation of macrophages.

Non-limiting examples of preferred immunosuppressive compounds include steroids (e.g., prednisone, methylprednisolone) and FTY720 (e.g., Shuurman et al., Transplantation 74:951-960, 2002; Droogan et al., Neurology 50:224-229, 1998). Additional exemplary immunosuppressive compounds include antibodies or compounds that block the production or functioning of mac-1, an antigen on macrophages known to be important for transmigration (e.g., Eslami et al., J. Vasc. Surg. 34:923-929, 2001, Shang et al., Eur. J. Immunol. 28:1970-1979, 1998), any compound that blocks monocyte chemoattractant protein (MCP-1) production or function or both (Ikeda et al., Clin. Cardiol. 25:143-147, 2002), antioxidants such as α-tocopherol (Devaraj et al., Nutr. Rev. 60:8-14, 2002; Terasawa et al., Biofactors 11:221-233, 2000), as well as compounds that block P-selectin, PSGL-1, VLA-4 or VCAM-1 activity (Huo et al., Acta Physiol. Scand. 173:35-43, 2001).

In addition, immunosuppressive compounds include, but are not limited to α-tocopherol, azathioprine, chlorambucil, cyclophosphamide, antibodies that bind to an IL-2 receptor or to CTLA4, methotrexate, mycophenolate mofetil, cyclosporine, agents that interfere with macrophage function, agents that inhibit P-selectin biological activity, agents that PSGL-1, VLA-4, VCAM-1, or Mac-1 biological activity.

By “intima” is meant the inner lining or inner layer of a vessel.

By “macrophage activity” is meant the ability to present foreign antigen to antigen-reactive lymphocytes and the ability to induce both the humoral and cell-mediated immune responses.

By “migration” is meant the movement of smooth muscle cells in vivo from the medial layers of a vessel into the intima.

By “mTOR inhibitor” is meant a compound that, alone or in conjunction with another compound, reduces the kinase activity of mTOR by at least 10% (more desirably, by at least 20%, 30%, 40%, 50%, 60%, 70%, or more) relative to the activity of mTOR in the absence of the compound. For example, rapamycin is an mTOR inhibitor—it forms a complex with FKBP-12, and this complex binds to and inhibits the kinase activity of mTOR. Other mTOR inhibitors are rapamycin analogs such as everolimus and temsirosimus (CCI-779), which are described herein.

By “neointimal hyperplasia” is meant an abnormal proliferation of smooth muscle cells after migration into the intima. Abnormal as used herein means division or growth of cells, but not cancer cells, that occurs more rapidly or to a significantly greater extent than typically occurs in a normally functioning cell of the same type. As a result of neointimal hyperplasia, a flow restricting lesion may occur diffusely throughout the graft or, more commonly, at focal sites near anastomoses or within the body of the graft.

By “peripheral vascular disease (PVD)” is meant a disorder in which the tissues, organs, or muscles do not receive the oxygen and other nutrients they require in order to function properly. PVD is usually caused by a gradual accumulation of plaque in the arteries causing the arteries to become narrow which restricts the flow of blood throughout the body.

By “pharmaceutically acceptable carrier” is meant a carrier that is physiologically acceptable to the treated mammal while retaining the therapeutic properties of the compound with which it is administered. One exemplary pharmaceutically acceptable carner substance is physiological saline. Other physiologically acceptable carriers and their formulations are known to one skilled in the art and described, for example, in Remington's Pharmaceutical Sciences, (20^{th} edition), ed. A. Gennaro, 2000, Lippincott, Williams & Wilkins, Philadelphia, Pa.

By “platelet derived growth factor receptor (PDGFR) biological activity” is meant any and all of the functions of the PDGFR. The PDGFR functions to elicit a mitogenic response as well as a chemotactic response in cells. Its functions can include: ligand binding which can include any of three dimeric forms of the PDGF ligand (AA, AB, or BB), receptor dimerization, autophosphorylation on a tyrosine residue, transphosphorylation of a substrate polypeptide or protein on a tyrosine residue, and recruitment of SH2 domain containing proteins. There are many standard assays for PDGFR biological activity known in the art and any of these can be used to assay a potential compound for its ability to inhibit PDGFR biological activity. Examples include cell proliferation assays, such as BrdU labeling and cell counting experiments; quantitative assays for DNA synthesis, such as ³H-thymidine incorporation; ligand binding assays and Scatchard plot analysis; receptor dimerization assays; and cellular phosphorylation assays (see, for example, Bioukar et al., J. Biol. Chem. 274:21457-63, 1999; Conway et al., Biochem. J. 337:171-7, 1999; Vignais et al., Mol. Cell Biol. 19:3727-35, 1999; Baxter et al., J. Biol. Chem. 273:17050-5, 1998; DeMali et al., Mol. Cell Biol. 18:2014-22; and Davies et al., Circ. Res. 86:779-786, 2000). One preferred assay for PDGFR biological activity is a phosphorylation assay using anti-phosphotyrosine antibodies (e.g., 4G10, Upstate Biotechnology Inc.). Immunoblots on whole cell lysates can be performed using this antibody to detect an overall increase in cellular tyrosine phospholylation. Tyrosine phosphorylation of specific substrates such as Src or p42/p44 MAP kinase can also be analyzed by immunoprecipitating the substrate protein followed by immunoblotting using an anti-phosphotyrosine antibody. Autophosphorylation of PDGFR itself is measured by immunoprecipitating PDGFR and immunoblotting with an anti-phosphotyrosine antibody. Each of the above-mentioned assays is quantitated and used to determine the effects of a potential inhibitor of PDGFR biological activity as compared to a control compound that does not affect PDGFR biological activity. Inhibition of biological activity depends on the assay being used, but generally connotes a reduction of at least 10% of the assayed activity, preferably at least 25%, more preferably at least 50%, and most preferably at least 75% of the assayed activity as compared to a control.

By “polytetrafluoroethylene graft (PTFE)” is meant a synthetic graft where fibers are woven into a mesh called Gore-Tex and made into a sleeve and flange and then attached to a vein at one end of the graft and an artery at the other end of the graft. This synthetic graft then provides access for the needles required for hemodialysis.

By “prevent or ameliorate” is meant a reduction in the narrowing of the vessel lumen diameter such that blood flow does not fall below values considered to be normal for the specific vessel. Clinicians or practitioners skilled in the art will be familiar with the normal values for blood flow for a specific vessel. It is preferred that the narrowing of the vessel lumen diameter is reduced by 20% or more, more preferably 30% or more and most preferably 50% or more. Surveillance of blood flow in dialysis can be accomplished using a transonic HD01 flowmeter (Transonics Inc., Ithaca, N.Y.).

By “prevent or ameliorate” can also be used in reference to neointimal hyperplasia and includes any decrease of 20% or greater (more preferably 50% or greater, most preferably 75% or greater) in the proliferation rate of vascular smooth muscle cells.

By “proliferation” is meant an increase in cell number, i.e., by mitosis of the cells. As used herein proliferation does not refer to neoplastic cell growth.

By “radiation therapy” is meant the use of directed gamma rays or X-rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

By “smooth muscle cells” is meant those cells derived from the medial layers of vessels and adventitial vessels. Characteristics of smooth muscle cells include a histological morphology (under light microscopic examination) of a spindle shape with an oblong nucleus located centrally in the cell with nucleoli present and myofibrils in the sarcoplasm. Under electron microscopic examination, smooth muscle cells have long slender mitochondria in the juxta-nuclear sarcoplasm, a few tubular elements of granular endoplasmic reticulum, and numerous clusters of free ribosomes. A small Golgi complex may also be located near one pole of the nucleus. The majority of the sarcoplasm is occupied by thin, parallel myofilaments that may be, for the most part, oriented to the long axis of the muscle cell. These actin-containing myofibrils may be arranged in bundles with mitochondria interspersed among them. Scattered through the contractile substance of the cell may also be oval dense areas, with similar dense areas distributed at intervals along the inner aspects of the plasmalemma.

By “effective amount” is meant an amount sufficient to prevent or ameliorate neointimal hyperplasia, venous stenosis, or vascular access dysfunction. It will be appreciated that there will be many ways known in the art to determine the therapeutic amount for a given application. For example, the pharmacological methods for dosage determination may be used in the therapeutic context.

By “thrombosis” is meant the formation or presence of a clot in the cardiovascular system that may be occlusive or attached to the vessel without obstructing the lumen.

By “tyrosine kinase activity” is meant the ability to catalyze the transfer a phosphate group from adenosine triphosphate (ATP) to a tyrosine residue on a substrate polypeptide or protein.

By “vascular access dysfunction” is meant a failure of the dialysis graft or the AV fistula. Dialysis graft failure is diagnosed by the loss of a thrill over the graft access site. A thrill is a palpable vibration accompanying a cardiac murmur and loss of a thrill can often indicate a loss of blood flow. Dialysis graft failure is also diagnosed by a loss of access blood flow as measured using a transonic HD01 flowmeter. Causes of vascular access dysfunction can include neointimal hyperplasia, venous stenosis, and thrombosis.

By “vascular stenosis” is meant a pathologic narrowing of a blood vessel.

By “venous stenosis” is meant a pathologic narrowing of a vein.

Other features and advantages of the invention will be apparent from the following description of the preferred embodiments thereof, and from the claims.

### BRIEF DESCRIPTION OF THE DRAWINGS

FIG. 1 is a photomicrograph showing PDGF-B chain staining in a human AV graft. The primary antibody used was a rabbit anti-human PDGF-B chain polyclonal antibody (Santa Cruz Biotechnology, Inc. sc-7878) at 1/200 dilution in PBS. Lower inset is the negative control.

FIG. 2 is a photomicrograph showing PDGF receptor β chain in a human AV graft. The primary antibody used was rabbit anti-human PDGF receptor β antibody (Santa Cruz Biotechnology, Inc. sc-339) at 1/50 dilution in PBS. Lower inset is the negative control.

FIG. 3 is a photomicrograph showing phosphorylated PDGF receptor β chain staining in a human AV graft. The primary antibody was rabbit anti-human p-PDGF receptor β chain (Tyr 751) antibody (Cell Signaling Tech. #2971) at 1/50 dilution in PBS. Lower inset is the negative control.

FIG. 4 is a photomicrograph showing PDGF receptor A chain staining in a human AV graft. The microwave method was used to retrieve antigen. The primary antibody used was rabbit anti-human PDGF receptor a polyclonal antibody (Santa Cruz Biotechnology, Inc. sc-338) at 1/200 dilution in PBS. Lower inset is the negative control.

FIG. 5 is a photomicrograph showing phospho-p70 S6 kinase (p-p70 S6 kinase) staining in a human AV graft. The primary antibody used was goat anti-human p-p70 S6 kinase polyclonal antibody (Santa Cruz Biotechnology, Inc. sc-338) at 1/100 dilution in PBS. Lower inset is the negative control.

FIG. 6 shows hematoxylin and eosin staining of a vein near a porcine AV graft showing: (A) slight thickening of the vein wall , and (B) a markedly thickened vein wall both secondary to neointimal hyperplasia; (C) higher magnification of neointimal lesion.

FIG. 7A shows an angiogram of venous stenosis taken during angiography of venous stenosis. The catheter was inserted into the graft and advanced to the vein-graft junction. An area of venous stenosis at this junction is shown as well as regions of post-stenotic dilatations on both sides of the stenosis (proximal and distal vein). Blood flow from the AV graft into the vein goes proximally (towards the heart) and distally (data not shown). FIGS. 7B-7D shows IVUS images of the venous side of AV graft at the time of graft harvest. FIG. 7B shows a demonstration of post stenotic veno-dilatation; FIG. 7C shows a demonstration of luminal narrowing at the graft vein junction, which is located at the 1 o'clock position; and FIG. 7D shows a demonstration of maximal luminal narrowing (diameter 3.1 mm) just distal to the graft-vein junction, resulting in 80.1% stenosis, as shown on the bottom left corner. FIG. 7E shows an example of 3D reconstruction of IVUS images in an artery performed by stacking 2D images (with the automated pullback). This allows quantitation of intimal volume (in addition to standard diameter and area measurements).

FIG. 8 is a photomicrograph showing immunohistochemistry for PDGF-B chain in the porcine AV graft lesion. The primary antibody used was rabbit anti-human PDGF-B chain polyclonal antibody (Santa Cruz Biotechnology, Inc. sc-7878) at 1/200 dilution in PBS.

FIG. 9 is a photomicrograph showing immunohistochemistry for PDGF receptor B chain. The primary antibody used was rabbit anti-human PDGF receptor B polyclonal antibody (Santa Cruz Biotechnology, Inc. sc-339) at 1/100 dilution in PBS.

FIG. 10 is a photomicrograph showing immunohistochemistry for p-p70 S6 kinase. The primary antibody used was goat anti-human p-p70 S6 kinase polyclonal antibody (Santa Cruz Biotechnology, Inc. sc-338) at 1/50 dilution in PBS. Upper insets in FIGS. 8 and 10 are negative controls. Upper set in FIG. 9 is higher magnification with primary antibody; lower inset is negative control.

FIG. 11 shows in situ hybridization analysis of MRB in placenta. FIG. 11A shows results with an antisense probe and FIG. 11B shows results with a sense probe.

FIG. 12 is a graph showing dose-response inhibition of hPDGF-BB (20 ng/ml) induced migration of HASOM cells by Gleevec (G) alone, rapamycin (R) alone and in combination (drug concentrations for G are in ng/ml and for R in pg/ml). First lane is without PDGF; all others are with PDGF. First two lanes have no drug added. The results are expressed as means±S.E.M. of triplicate readings.

FIG. 13 is a graph showing dose-response inhibition of PDGF (20 ng/ml) induced migration of porcine cells by Gleevec G (drug concentrations are in ng/ml). Last lane is with DMSO at a concentration that is in the highest Gleevec data (lane 6). The results (number of migrated cells) are expressed as means±standard error of the mean (S.E.M.) of triplicate readings on the y-axis.

FIG. 14 is a schematic diagram.

### DESCRIPTION OF THE INVENTION

The invention generally features the inventor's discovery that (i) platelet derived growth factor receptor (PDGFR) inhibitor compounds, such as N-phenyl-2-pylimidine compounds (e.g., imatinib mesylate), inhibit the biological activity of the PDGFR, and (ii) mTOR inhibitors prevent neointimal hyperplasia in vivo. Accordingly, these compounds are useful individually or together in treating AV graft failure.

### Events Leading to Graft Failure

Graft failure resulting from neointimal hyperplasia is a major health problem for patients undergoing hemodialysis as well as those undergoing by-pass graft surgery as a treatment for advanced PVD. The stenotic lesion that leads to graft failure is typically characterized by three key events; migration of vascular smooth muscle cells into the intima; followed by proliferation of these vascular smooth muscle cells, and subsequent production of extracellular matrix.

This combination of migration, proliferation, and extracellular matrix production results in neointimal hyperplasia and stenosis. In addition, neo-vascularization occurs both in the intimal and the adventitial regions of the vessel and the presence of macrophages has also been noted.

### Distinctions Between Arterial and Venous Neointimal Hyperplasia

Much of what is known about venous neointimal hyperplasia is derived from studies of experimental arterial models, such as coronary artery disease and angioplasty. There are several key differences between arterial neointimal hyperplasia and the venous neointimal hyperplasia associated with vascular access dysfunction. For example, venous stenosis associated with vascular access dysfunction occurs in a uremic environment that is not present in arterial models. In addition, in the arterial wall, there is a distinct boundary between the intimal and medial layers of the vessel, which is not present in the vein. Furthermore, the neointimal hyperplasia associated with the PTFE graft lesion is chronic in nature and is characterized by a continuous proliferation of vascular smooth muscle cells and of extracellular matrix deposition.

### Pathogenic Mechanisms of Intimal Hyperplasia

Based on studies of non-AV graft neointimal hyperplastic lesions, numerous pathogenetic mechanisms have been proposed for the development of intimal hyperplasia in AV grafts. These mechanisms are typically initiated by upstream events including injury to the vessel wall, immune/inflammatory events, and hemodynamic events, which all funnel into downstream events, including vascular smooth muscle cell migration and proliferation, extracellular matrix production, and possibly angiogenesis. In addition, it now appears that de-differentiation, or a lack of differentiation of the vascular smooth muscle cells in the neointima may play a role in the pathogenesis of the lesion. Cell types other than the vascular smooth muscle cells are also likely to be involved in the initiation or maintenance of the neointimal lesion. These include monocytes that migrate into the wall of the vessel, endothelial cells that provide neovascularization within the neointima and the adventitia, and platelets that release growth factors such as PDGF and serotonin.

### PDGF

PDGF is a homo/heterodimeric ligand that can be found in three forms: AA, AB, and BB. The PDGF receptor, which is a tyrosine kinase involved in many signaling pathways, can be found in two forms: alpha (α) and beta (β). Each receptor type shows specificity for each ligand subtype with the PDGFRα able to bind all three ligands and the PDGFRβ only able to bind the BB subtype.

### Imatinib Mesylate

Imatinib mesylate (Gleevec™) is an N-phenyl-2-pyrimidine derivative that has been shown to inhibit the Bcr-Ab1 tyrosine kinase activity as well as c-kit, c-Ab1, and PDGFR tyrosine kinase activity. Clinical trials using imatinib mesylate to treat patients with chronic myelogenous leukemia have shown a great deal of success.

N-phenyl-2-pyrimidine derivatives, such as imatinib mesylate, function as tyrosine kinase inhibitors that have proven very effective in the treatment of diseases such as chronic mylegenous leukemia. While the success of imatinib mesylate in the treatment of chronic myelogenous leukemia seems unrelated to vascular access dysfunction, the ability of imatinib mesylate to inhibit PDGFR biological activity is useful in the treatment of patients suffering from graft failure due to neointimal hyperplasia. Without being bound to any particular theory, it may be that PDGFR has a mitogenic role in neointimal hyperplasia. By preventing mitogenic signaling from the PDGFR, the hyperproliferation of vascular smooth muscle cells is inhibited. This inhibition results in a decrease in neointimal hyperplasia thereby preventing or reducing graft failure related to vascular access dysfunction or by-pass graft treatment of PVD.

Imatinib mesylate functions through inhibition of the ATP binding site of several tyrosine kinases including: BCR-ABL, c-kit, c-Ab1 and PDGFR. By blocking the ATP-binding site, this chemical compound can prevent the transfer of a phosphate group from ATP to a tyrosine residue on a substrate protein or polypeptide.

### Rapamycin

Rapamycin is a cyclic lactone produced by Streptomyces hygroscopicus. Rapamycin forms a complex with the immunophilin FKBP-12. This complex binds to and inhibits the mammalian kinase target of rapamycin (mTOR), a kinase that is required for cell-cycle progression.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. Nos. 5,118,678; 5,508,399; 5,516,780; 5,519,031; 5,530,007; 5,530,121; 5,532,255; 5,550,122; 5,559,112; 5,559,120; 5,559,227; 5,567,709); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. Nos. 5,258,389; 5,665,772, 6,440,990); and deuterated rapamycin (U.S. Pat. Nos. 6,342,507; 6,503,921; and 6,710,053). Additional rapamycin analogs are described in U.S. Pat. Nos. 5,169,851; 5,202,332; 5,362,718; 5,521,194; 5,525,610; 5,541,191; 5,541,192; 5,559,122; 5,563,145; 5,597,715; 5,637,590; 5,648,361; 5,661,156; 5,677,295; 5,712,129; 5,728,710; 5,776,943; 5,780,462; 5,912,253; 5,922,730; 5,955,457; 5,985,890; 6,015,815; 6,200,985; 6,328,970; 6,329,386; 6,331,547; 6,399,625; 6,432,973; and 6,680,330. The compounds described in each of the foregoing patents and publications are hereby incorporated by reference.

One particularly useful rapamycin analog is everolimus (40-O-(2-hydroxyethyl)rapamycin; CERTICAN™; Novartis), an immunosuppressive macrolide that is structurally related to rapamycin. Methods of making everolimus are described in U.S. Pat. No. 5,665,772, hereby incorporated by reference.

Rapamycin is currently available for oral administration in liquid and tablet formulations. RAPAMUNE™ liquid contains 1 mg/mL rapamycin that is diluted in water or orange juice prior to administration. Tablets containing 1 or 2 mg of rapamycin are also available. Rapamycin is preferably given once daily as soon as possible after transplantation. It is absorbed rapidly and completely after oral administration. Typically, patient dosage of rapamycin varies according to the patient's condition, but some standard recommended dosages are provided below. The initial loading dose for rapamycin is 6 mg. Subsequent maintenance doses of 0.5-2 mg/day are typical. Alternatively, a loading dose of 3 mg, 5 mg, 10 mg, 15 mg, 20 mg, or 25 mg can be used with a 1 mg, 3 mg, 5 mg, 7 mg, or 10 mg per day maintenance dose. In patients weighing less than 40 kg, rapamycin dosages are typically adjusted based on body surface area; generally a 3 mg/m²/day loading dose and a 1 mg/m²/day maintenance dose is used.

### PDGF Pathway is Activated in Failed Human AV Grafts.

Failed human AV grafts were removed from patients and analyzed for immunohistochemical evidence of neointimal hyperplasia and expression of proteins related to the PDGFR signaling pathway as follows.

AV graft specimens were fixed in 10% formaldehyde and 6 μm paraffin embedded sections were made, dewaxed in xylene and rehydrated in alcohol. Samples were treated with Proteinase K for 15 minutes to unmask antigens, followed by two washes with absolute alcohol and phosphate buffered saline (PBS) (0.9% NaCl in 10 mM sodium phosphate buffer, pH 7.6). They were then incubated for 15 minutes in 1% H₂O₂ in absolute methanol to block endogenous peroxidase activity, followed by two washes with PBS, and then incubated with blocking solution (5% serum derived from the same species in which the secondary antibody was produced, 1% BSA in PBS) for 30 minutes to reduce nonspecific binding of secondary antibody, followed by two PBS washes.

The sections were then incubated with a primary antibody for 16 hours at 4° C. The primary antibodies included antibodies against PDGF A chain, PDGF B chain, PDGFRα, PDGFRβ expression, and antibodies that recognize a form of PDGFRβ that has been phosphorylated at a site relevant for signaling in the PI3K/Akt/mTOR pathways. After washing with PBS, the sections were treated with an HRP conjugated goat anti-rabbit secondary antibody at 1/200 dilution in PBS (Dako Po448) for 45 minutes or with a rabbit anti-goat secondary antibody at 1/200 dilution in PBS (Dako Po160). After a PBS wash, the color reaction was developed with diaminobenzidine (Dako) and counterstained with hematoxylin. Finally, the sections were dehydrated in alcohol and mounted in premount. Negative controls were performed by omitting primary antibody.

For PDGF-B immunohistochemistry the method described above was followed except that microwave treatment was used to retrieve antigen, as follows. Antigen retrieval buffer contained 9 ml of citric acid solution (21.01 citric acid in 1 liter of distilled deionized H₂O) and 41 ml of Na citrate solution (29.41 g Na citrate in 1 liter of distilled deionized H2O) in 500 ml of dH₂O was heated to boiling. The deparaffinized samples were placed in the solution and heated in a microwave for 6 minutes. The solution was cooled at room temperature for 10 minutes. The samples were then washed twice in PBS.

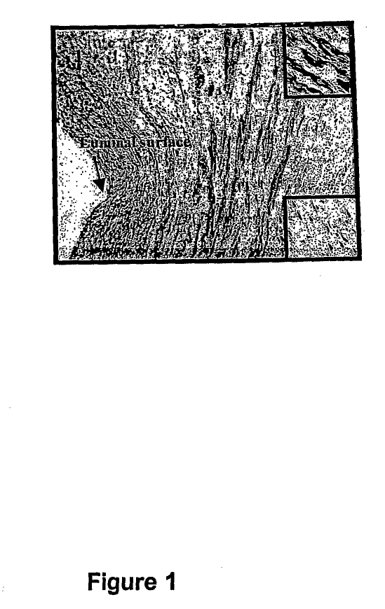

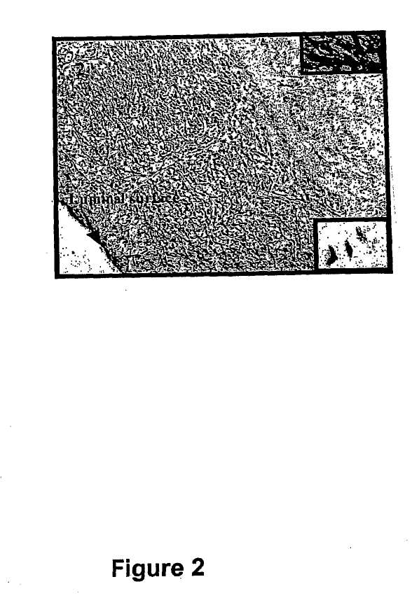

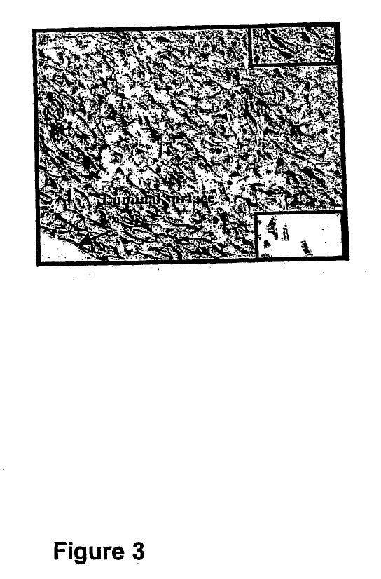

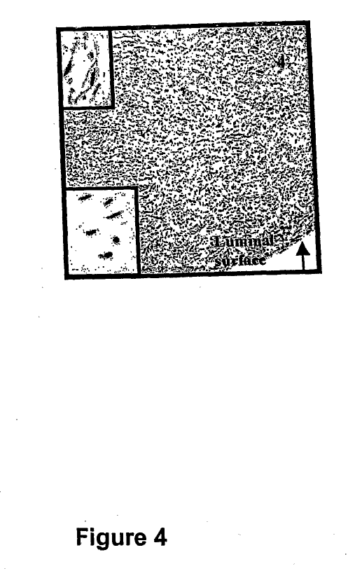

Striking expression of PDGF A chain, PDGF B chain (FIG. 1), PDGFRβ (FIG. 2), and PDGFRA (FIG. 4), and was observed in the intima of the failed human grafts providing evidence that the PDGF pathway is activated in such grafts. Antibodies that specifically recognize a form of PDGFRβ that has been phosphorylated at a site relevant for signaling in the PI3K/Akt/mTOR pathways demonstrated specific phosphorylation of the PDGFRβ at that site (FIG. 3). These results indicate that both PDGFR and PI3K signaling pathways are upregulated in failed human AV grafts. We have not yet assessed if the staining is in myofibroblasts or VSMCs based on desmin/a smooth muscle actin (SMA) positivity.

### Pig AV Graft Lesions Express PDGF Signaling Molecules

AV Fistula Surgery

Yorkshire pigs weighing 40-45 kg were given aspirin, 325 mg by mouth, once per day starting the day before surgery and continuing throughout the study period. Anesthesia was induced with ketamine 15 mg/kg and maintained with isoflurane administered through an endotracheal tube. A single dose of heparin (250 U/kg), buprenex (0.03 mg/kg), and cephazolin (35 mg/kg) was given preoperatively. The femoral vein and artery were surgically exposed on both sides. A loop arterio-venous fistula was created bilaterally using 6 mm internal diameter (ID) PTFE grafts, 6.5 cm in length (Impra Bard10S06) between the femoral artery and femoral vein by end-to-side anastamoses with 7-0 and 6-0 polypropylene sutures on the venous and arterial sides, respectively.

Baseline angiography was then performed as described below. The fascia and skin was closed using 2-0 vicryl and 4-0 Polyglycolic acid Dexon S homopolymer of glycolic acid (PDS) sutures, and the pigs were allowed to recover.

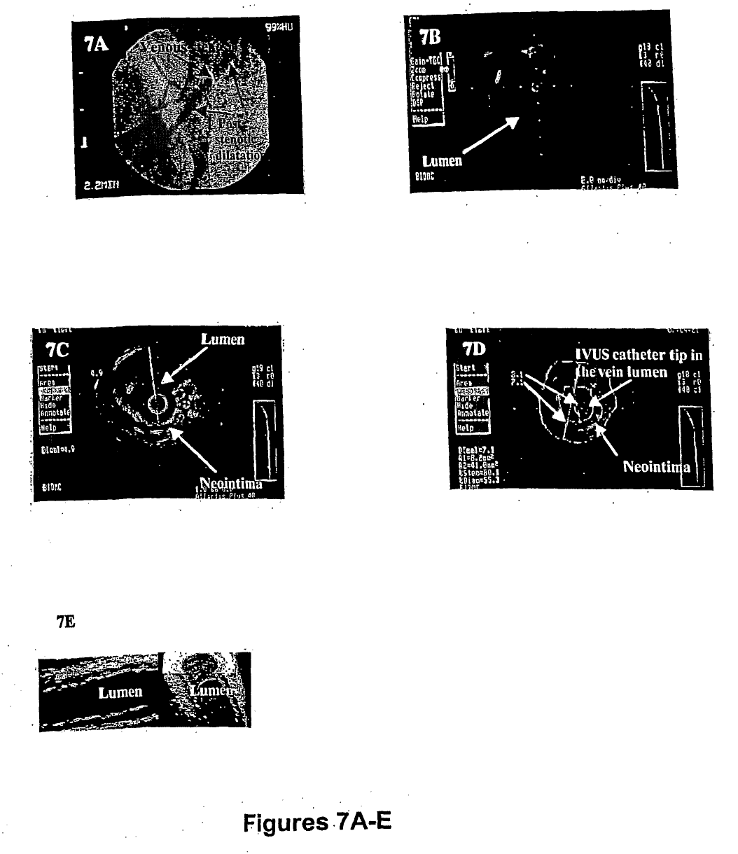

Fentanyl patches (2 ug/kg) were used for 72 hours for post-operative analgesia. Graft patency was assessed by auscultation immediately following surgery, at 1 week, and then at twenty-eight days (at the time of graft harvest). Grafts patent at 28 days showed roughly 70-90% maximal luminal area stenosis, as assessed by IVUS (see below and FIG. 7).

Graft Harvest

Twenty-eight days after graft placement, pigs were anesthetized as described above and graft patency and noeintimal thickness were assessed using intravascular ultrasound (IVUS) and angiography (as described below) prior to graft harvest. The grafts and surrounding femoral vessels were carefully dissected out, and femoral vessels ligated proximally and distally at least 2 cm away from the anastomosis and excised. The AV graft specimen were pressure fixed in 10% formalin for histopathological analysis (in all pigs). The pigs were sacrificed by injection of a high-potassium solution.

Porcine Lesion Histology

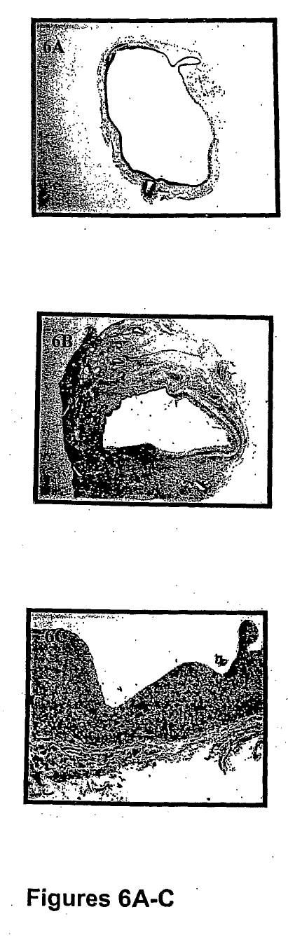

6 μm paraffin embedded sections were made. Hematoxylin and eosin staining was performed on the tissue sections for gross histological examination using standard methods. FIG. 6 shows that the porcine lesion is indistinguishable from the neointimal hyperplasia lesions seen in human grafts.

Assessment by Angiography

X-ray cine angiography was performed just after graft placement and at the time of graft harvest. The angiography procedure was carried out as follows. A 20-gauge angiocath was inserted into the AV graft and RENOGRAFIN-76, a high atomic weight contrast agent, was injected in different incidences for quantitative morphometric image analysis of AV graft lumen (antero-posterior, 30° and 45° right anterior oblique, 30° and 45° left anterior oblique images were obtained). After completion of angiography on one side, another angiocath was inserted into the other graft and angiography was performed. FIG. 7A shows angiographic data on a typical graft (at 28 days) clearly revealing severe stenosis and post-stenotic dilatation on both sides of the stenosis that occurs at the vein/graft anastomosis. Note that blood flows from the graft into the femoral vein and goes in both directions, i.e., towards the heart and away from it in the femoral vein.

Assessment by Intravascular Ultrasound (IVUS)

We performed IVUS at the time of graft harvest on day 28 as follows. Intravascular ultrasound (IVUS) catheters are tiny medical devices that, when inserted into a person's coronary arteries, reflect images from inside the coronary arteries to an attached console A 0.014″ guide wire (Choice PT EX wire, Boston Scientific, Mass.) was introduced into the femoral vein about 10 cm distal to the AV graft junction and advanced proximally. A 3.2 French 40 MHz IVUS catheter (Atlantis catheter, CVIS, Boston Scientific Corporation, Maple Grove, Mn.) was delivered over the guide wire and positioned in the femoral vein proximal to graft vein junction. Automated pullback was then performed at the rate of 0.5 mm/sec while recording IVUS images. The CVIS system in use in the laboratory offers resolution of 200 microns which is significantly superior to quatitative angiography and enabled us to make luminal and vessel wall measurements. As noted earlier, the main advantage of IVUS is an accurate cross-sectional area assessment and the ability to measure the area and volume of the intimal lesion. Images and loops were analyzed offline. Preferably, these are analyzed by an investigator blinded to treatment assignment. The IVUS images were analyzed for lumen cross sectional area and thickness of neointima. The images were collected and a typical example is shown in FIGS. 7B-7D. Note the typical 80% maximal stenosis area shown.

### PDGF Signaling Molecules are Expressed in Pig AV Graft Lesions

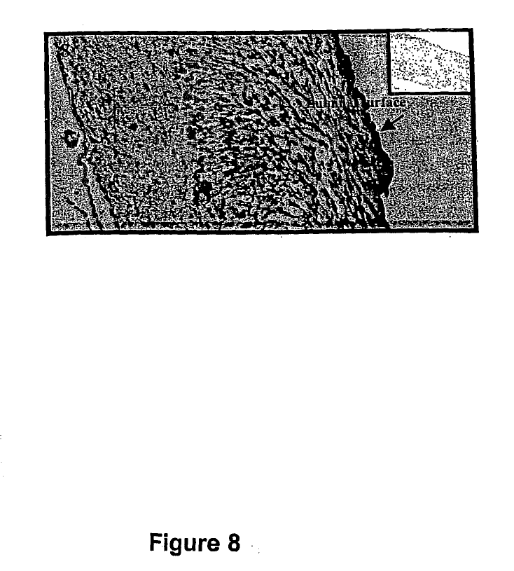

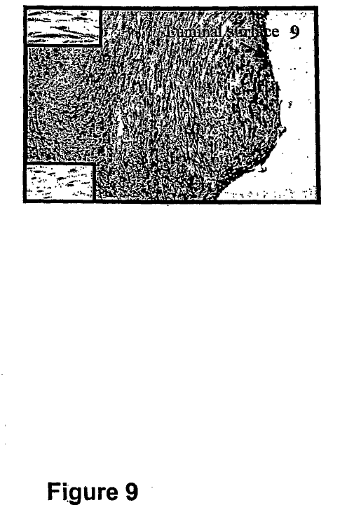

Immunohistochemistry was performed on pig AV graft lesions as described above for the human samples except that the microwave method was used to retrieve antigens. The samples were incubated in 3% H₂O₂ in absolute methanol for 15 minutes to block endogenous peroxidase activity and then the samples were incubated with Cyto Q background buster (Cat no-NB 306, Innovex Biosciences, CA), a signal-to-background booster solution for 30 minutes to diminish background staining. The antibody against the Tyr751 phosphorylated PDGF-β receptor did not detect signal in pig tissue, presumably due to pig-human β receptor sequence differences around the tyrosine 751 residue. However, the phospho p70 S6 kinase antibody did cross-react in the pig and provided evidence of MTOR activation (driven by PDGF or Ang-II or perhaps other cytokines). These data are shown in FIGS. 8-10 and are strikingly similar to the human immunohistochemistry data (FIGS. 1-5).

These results demonstrated that the porcine model shows the classic neointimal pathology observed in human AV grafts. Moreover, molecules involved in the PDGF signaling pathways were indeed expressed in the porcine AV graft lesion, thereby molecularly validating that the porcine model parallels the human situation and is useful to investigate therapeutic methods for blocking PDGF signaling.

Cytokine Expression in Porcine AV Grafts

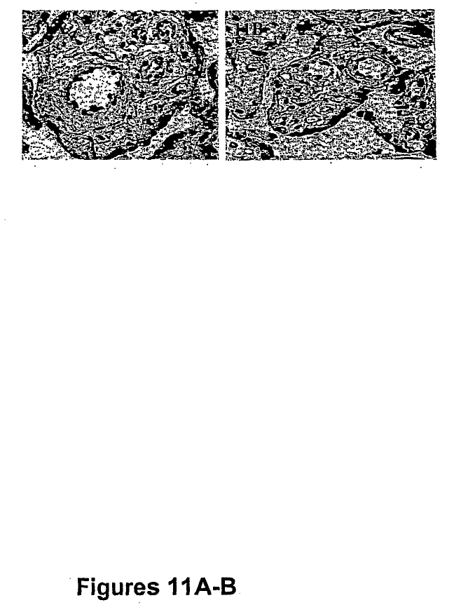

In situ methods enable us to determine those cells that produce PDGF and thus complement the antibody studies. In situ analysis is performed using a non-radioactive method (see St Croix et al., Science, 289: 1197-1202, 2000). Briefly, frozen tissue sections are fixed in 4% paraformaldehyde, permeabilized with pepsin, and incubated with RNA sense and antisense probes (300 ng/ml) (previously generated using digoxygenin RNA labeling reagents) overnight at 55° C. For signal amplification, a horse radish peroxidase (HRP) rabbit anti-digoxygenin antibody (DAKO, Carpinteria, Calif.) is used to catalyze the deposition of biotin-Tyramide (GenPoint Kit, DAKO). Further amplification is achieved by adding HRP rabbit anti-biotin and then alkaline-phosphatase rabbit anti-biotin. Signal is detected with AP substrate Fast Red TR/Napthol AS-MX, a precipitating substrate for the detection of alkaline phosphatase activity in immunochemistry, (Sigma, St Louis, Mo.) and cells were counter stained with hematoxylin. Such staining was carried out in placenta (FIG. 11) with a probe for a gene called MRB (magic roundabout). Similar methods will be used with PDGF and PDGF receptor probes.

### Gleevec and Rapamycin Inhibit Smooth Muscle Cell Migration

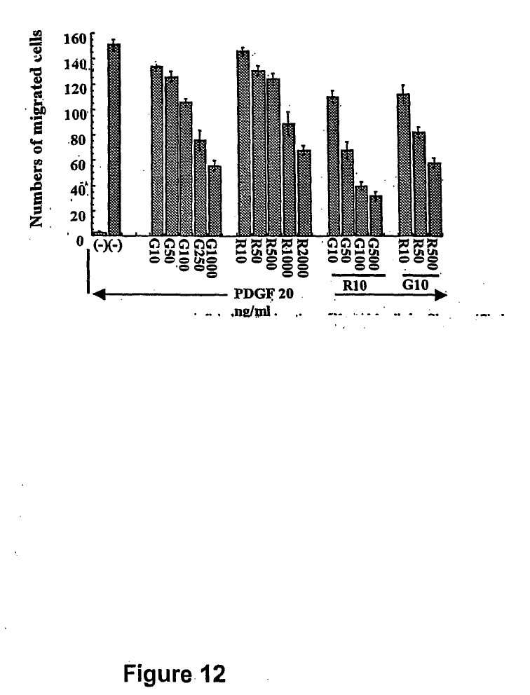

In vitro studies were performed to examine the dose response of Gleevec and rapamycin on human aortic vascular smooth muscle cell (HASOM cells) migration using a 24-well Boyden-chamber (Coming Costar Corp, Cambridge, Mass.) as follows. HASOM cells were cultured to 70% confluence, then treated with media containing 1% fetal calf serum and Gleevec alone, rapamycin alone, or Gleevec and rapamycin in combination. The concentrations of the various compounds used are indicated in FIG. 12. The cells were then cultured in the presence of these agents for forty-eight hours prior to the migration assay. The cells were harvested in 0.05% trypsin and resuspended in M-199 media and seeded at a concentration of 4000 cells per well with Gleevec and/or rapamycin. Each well contained an insert with a polycarbonate filter having 8 μm pores. In preparation for the migration assays, the membranes were coated with 0.1% gelatin for 8 hours, aspirated, and allowed to dry for 2 hours.

The M-199 media containing 0.5% BSA and 20 ng/ml human PDGF-B (hPDGF-BB) was placed in the bottom Boyden chamber. Cells were incubated at 5% CO₂ at 37° C. for twelve hours and the cells lying on the upper surface of the membrane were scraped off. The insert membranes were removed and the cells on the lower surface of each membrane were stained with hematoxylin. Cell migration was then quantified by counting the cells in five random high-power fields on the lower surface of the membrane. All assays were run in triplicate. The results of this assay are shown in FIG. 12.

We found that Gleevec alone, rapamycin alone, and a combination of Gleevec and rapamycin potently inhibited PDGF induced migration of HASOM cells in a dose dependent manner (FIG. 12). The combination of Gleevec and rapamycin provided an additive, if not synergistic, effect (FIG. 12). Note that the effective synergistic concentrations of rapamycin are well below those that typically cause immunosuppression (roughly 5-15 ng/ml). The maximum inhibition observed is on the order of 70-80%. We suspect that the presence of small amounts of serum in the assay might have introduced factors whose signals were not blocked by Gleevec or rapamycin. Extrapolating to the microenvironment of the AV graft, increased inhibition would likely be observed if rapamycin and/or Gleevec were administered together with drugs, such as ACE inhibitors, that block the effects of cytokines, such as Ang-II. Gleevec and rapamycin can be delivered either systemically or locally. Thus, this migration assay is useful for identifying therapeutic compounds of the invention. Useful compounds are then tested in animal models, as described below.

### Gleevec Inhibits PDGF Induced Migration of Cells of Porcine Origin

While rapamycin was known to inhibit the activation of mTOR in porcine cells, we tested the effects of rapamycin on PDGF signaling in porcine cells. We isolated cells of porcine origin from a primary explant of porcine carotid artery using standard methods as follows.

A porcine carotid artery sample was obtained from a control animal at the time of sacrifice using aseptic technique. The sample was placed in PBS and left on ice. The arterial sample was opened with a longitudinal incision in a tissue culture hood. The endothelial layer was scraped off and the adventitia was peeled off from the outer surface of the vessel wall. The sample was cut into 0.5×0.5 cm pieces and placed in culture media (DMEM with 10% FBS, ampicillin, and gentamicin) with the endothelial surface facing down. The media was changed every two days. The cells began migrating from the tissue from day 5 onwards and were confluent by day 8. The cultured cells were collected after trypsinisation and the experiments described below were performed on second passage cells.

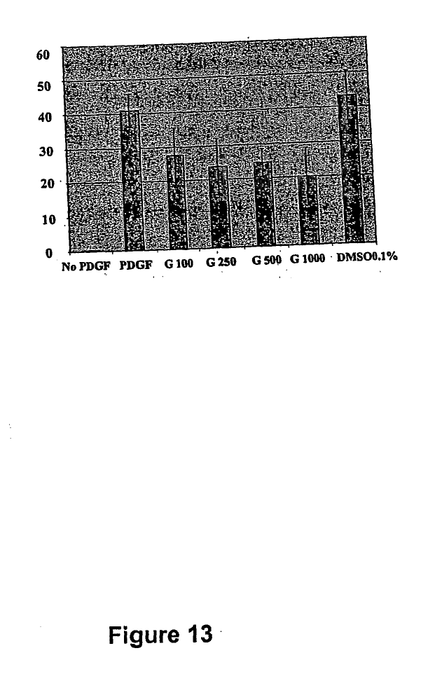

The isolated porcine cells were tested in migration assays carried out using the methods described above. These cells showed an excellent dose response to PDGF (20-fold above background) when HPDGF-BB at 20 ng/ml was used. Importantly, we found that Gleevec in inhibited PDGF-B induced migration of these porcine cells a dose dependent manner. The porcine dose response curve resembled that of human VSMCs (FIG. 13).

### Measurement of Compound Efficacy in an in Vitro Smooth Muscle Cell System

Smooth muscle cells were induced to migrate by adding PDGF-BB to cultures of cells in vitro as described above. Gleevec was added to the migrating smooth muscle cells and inhibited migration. Rapamycin was also added to the migrating smooth muscle cells and also resulted in an inhibition of migration. Importantly, when Gleevec and rapamycin were added together as a combination, an enhanced inhibition of proliferation migration was detected as compared to the addition of either drug alone.

### Efficacy of Gleevec and Perindopril

ACE inhibitors are commonly used in patients on hemodialysis. While Ace inhibitors alone have not been found to be effective in maintaining graft patency, as detailed above, we report herein that ACE inhibitors are useful in treating neointimal hypelplasia. Therefore, Gleevec is administered in combination with perindopril and its effects are assayed as follows.

Fifty-two pigs receive each of four treatments (e.g., Gleevec; perindopril; Gleevec and perindopril in combination, and placebo). Drug treatment is administered 4 days before AV graft placement (day-4). Trough drug levels are obtained on the day of surgery (day 0), day 7, and day 28. Animals with complete thrombosis of both AV grafts on day 7 are considered surgical failures and are replaced. Graft patency is assessed by ausculation and doppler flow. Animals with a single viable graft are retained unless they show distress from the thrombosis, in which case they are sacrificed and replaced.

Stenosis is assayed using IVUS on day 28. Pigs having 75% or more stenosis in both grafts on day 28 are sacrificed. The remaining animals are assayed for stenosis every 7 days until stenosis reaches 75% in both grafts, at which time the remaining animals are sacrificed. Groups are compared day 28 and at the time when 75% stenosis is reached. This will allow us to study the long-term progression of stenosis. Treatments that reduce the degree of stenosis by 10%, 20%, 30%, 40%, 50%, 60%, 70%, 80%, 90%, or 100% are taken as useful in the invention.

Differences in the treatment arms may be accentuated after the 28 day time points. This will allow us to identify clinically important differences between the three treatments on the degree of stenosis, and between the treated groups and the control group.

Immunohistochemical analyses of the graft lesion are carried out to assay for cell phenotype and for cytokine and intracellular signaling. Gleevec and perindopril levels in the blood are assessed, as will any obvious evidence of toxicity (CBC), necrosis in the graft region (perhaps due to the drug), or infection at the graft site.

The extent of maximal area graft/vein stenosis at 28 days and subsequently (described above) is assessed by IVUS as detailed above.

IVUS

The IVUS measurements are carried out on veins over a 2-3 cm distance on either side of the graft/vein junction as this region is where the neointimal lesion occurs. In addition, IVUS is performed in the graft if angiography shows stenosis there. The degree of stenosis is quantitated i) by determining the minimal luminal area; ii) by measuring the maximal percent area that is stenosed; iii) by producing a 3-dimensional reconstruction of the lesion and then quantifying the volume occupied by the lesion within the graft, divided by the total volume as an index of stenosis; or iv) by determining the total blood flow (cc/min) in the graft.

IVUS is conducted on day 0 in two animals in each group to ascertain a baseline. This will allow us to determine whether there is considerable variability in the vein size of the pig. If there is variability, it will further encourage us to consider determining the % area stenosis rather than the absolute open area at the 28 day time point. Angiography is perfonned on day 0 and at day 28. The will allow us to quickly assess the condition of the graft and the anastomotic site.

The following parameters IVUS are recorded for baseline at day 0, at 28 days and on subsequent measurements: luminal diameter and cross sectional area along the vein and in the graft so as to obtain the % NIH area at the site of maximal stenosis and total NIH volume (3D reconstruction—see example in FIG. 7E).

Blood Gleevec levels, cellular phenotype, cytokine and intracellular signaling analysis, and histopathology of the graft and vein are determined at the time of sacrifice as follows.

Gleevec Blood Levels

Mass spectrometry is used to measure blood Gleevec levels down to a level of detection of about 30 ng/ml using standard methods (see, for example, Wolff et al. Blood. Feb. 20, 2003 (epub ahead of print)). Gleevec levels will allow us to determine whether there is any correlation between Gleevec levels and the extent of stenosis reduction obtained with Gleevec treatment

### Cellular Phenotyping

The number and location of smooth muscle cells and myofibroblasts as well as macrophages and endothelial cells is determined using antibodies against αSMA, desmin, vimentin, macrophage surface antigens, and von willebrand factor (vWF) as described in Kelly et al., (Kidney Int, 62: 2272-2280, 2002.

PDGF blockade may result in an alteration in the number of cell types, in their relative ratios, or in their location in the lesion. For example, it would be interesting to assess the number of VSMCs and myofibroblasts in the lesion: PDGF signaling inhibitors may decrease the latter without affecting the former thus leading to less neointimal hyperplasia. Depending on our initial findings, we may quantitate the degree of cellular proliferation and apoptosis in each cell type using BrDU and a standard tunnel assay as described in Kelly et al., (Kidney Int, 62:2272-2280, 2002).

Chtokine and Intracellular Analysis

Cytokine and intracellular signaling analysis is carried out using antibodies that detect porcine β receptor phosphorylation or immunoprecipitation of cell extract with a β receptor antibody followed by use of a generalized phosphotyrosine antibody to determine whether Gleevec and/or perindopril have effected the signaling pathways.

If the target has been “hit”, and if there is still significant NIH, then intracellular pathways are evaluated for activation following drug therapy. Our attention will focus on the PI3K-Akt-mTOR-p70 S6 kinase pathway and on MEK-ERK ½ activation, since both are downstream of PDGF and other cytokine signaling pathways and are known to be important in migration/proliferation of VSMCs. In particular, we will use phospho ERK ½ antibodies for immunohistochemistry or western blot analysis in pig tissue. Alternatively, immunoprecipitation are carried out followed by western blot analysis utilizing a generalized phosphotyrosine antibody on NIH tissue to ascertain activation. The phospho p70 S6 kinase antibody can also be used for immunohistochemistry or western analysis, to assess mTOR activation. For example, if PDGF blockade by Gleevec resulted in blockade of ERK ½ activation, but not of mTOR activation, it is likely that cytokines other than PDGF are activating mTOR and that targeting mTOR activation by rapamycin or another MTOR inhibitor is useful. In addition, perindopril could be used in the methods of the invention to diminish mTOR activation by Ang-II as levels of Ang-II are reduced by perindopril.

In addition to these intracellular signaling events, immunohistochemistry, in situ methods, and western analysis are used to analyze the expression in the lesions of the following cytokines: bFGF, MCP-1, which should be decreased if PDGF signaling is blocked, VEGF, and ET-1. Comparison are made between our four study groups with a semi-quantitative scoring scale, for example, from 0-5 for most intense expression. These cytokines are known mitogens/motogens for VSMCs, endothelial cells and moncytes. Also, drugs to block several of these cytokines (e.g. ET-1 and VEGF receptor antagonists) or the previously noted intracellular pathways (MEK-ERK inhibitors) are available and, at least for local delivery, may be quite useful in the methods of the invention.

### Porcine Primers

The appropriate pig cDNAs are identified using PCR primers selected from conserved regions (e.g., regions having at least 60%, 75%, 80%, 90% or 100% nucleic acid sequence similarity or identity) as determined by aligning the cDNAs from mouse, human, rat, and lower organisms. The PCR products are obtained by RT-PCR of porcine RNA derived either from cell culture of porcine VSMCs or from RNA made from a pig aorta or from a pig neointimal lesion. These sources should be adequate to provide the desired clones. Of course, all the clones are sequenced and should show a high degree of homology to the human and rodent clone sequences.

### Statistical Considerations.

A total of 13 animals/group is used with surgical failures replaced as required. Gleevec will likely have a marked effect on stenoses as compared to controls receiving no drug therapy. In the control group, we expect stenosis of approximately 80% with a standard deviation of 10-15% based on our experience when stenosis at the 28 day point is evaluated. The sample size for the overall experiment is driven by the desire to detect a difference between Gleevec and the combination group of Gleevec+perindopril. The sample size selected will provide 90% power to detect a difference of 20% stenosis (at the fixed time of 28 days) between the Gleevec and combination group, or 80% power to detect a difference of 17.5% stenosis (significance level of 0.05, two-sided, using a t-test, using 15% as the estimated within group standard deviation (SD)). Note that this is an extremely conservative power calculation, intended to demonstrate that we will have adequate power to detect clinically important differences, as the analysis described below would be expected to have far higher power to detect differences between the two groups.

Approach to Data Analysis

Although the experiment is a 2×2 factorial experiment (Gleevec yes or no x perindopril yes or no), we describe the analysis in terms of a two group comparison between the Gleevec only group and the combined Gleevec +perindopril group; the approach described here can easily be extended to the full factorial analysis, if desired. We would begin with a graphical examination of the results within each animal, plotting stenosis over time separately for each of the two grafts in an animal. These profile plots could be extended to plot results from multiple animals (within a group) or summary data (e.g., boxplots) for all groups over time. This is an essential step to gain some insight into the data. Although we could analyze each time point individually using basic techniques (e.g. a t-test or Wilcoxon Rank Sum test using the average of the two measurements of stenosis, or a Fisher's Exact test if results are classified into a binary result such as “significant stenosis: yes or no”), as we should have serial measurements from two sites per animal, with different measurement schedule in different animals, it is much more efficient statistically to use modern techniques for longitudinal data analysis. Alternatively, a mixed models analysis of variance (it stenosis or a transform appears to be distributed as a normal variable), or a generalized estimating equation is used. Such models allow us to incorporate serial measurements within an animal over time, allow us take into account that the two grafts within an animal are correlated, and allow for varying intervals between measurements and potentially different measurement schedules for different groups. We can incorporate treatment group by using indicator variables, and extend the analysis to a full factorial analysis by incorporating a Gleevec×perindopril×time interaction term. Additional potential factors (e.g., cytokine levels, or degree of phosphorylation of intracellular signaling molecules such as p70 S6 kinase, serum Gleevec levels) are easy to incorporate in this general modeling framework. This approach allows data from all animals to be used, even if some measurements are missing or incomplete for an animal. Thus, even if the experimental plan is modified after the first two-three blocks of animals, the data from these animals is included in the overall analysis of the experiment.

### Gleevec and Rapamycin Drug Delivery Systems

Loading Dose

Based on the IC50 of rapamycin for blocking VSMC migration (1-5 ng/ml), we expect that a 1-2% loading dose is used, whereas for Gleevec, a 30-50% loading dose is necessary given Gleevec's IC50 (250-500 ng/ml for Gleevec).

Using the IC50 in vitro it is possible to extrapolate the amount of each drug required for daily release. For example, rapamycin eluting stents elute approximately 185 μg of rapamycin over approximately a 30 day period (74). A perivascular device, such as a film or a suture, wrapped around 4 cm of vein (2 cm on either side of the graft/vein anastmosis) and 1 cm of graft tissue having a weight between 200 and 400 mg, we estimate that a 0.05% to 0.1% loading dose is adequate. Desirably, 1-2% (a considerably higher number than 0.05% to 0.1%) dose is used for rapamycin.

For Gleevec, desirably a 30-50% loading dose is used. This dosage is based on routine methods for the use of other drugs, such as verapamil. When verapamil was used perivascularly to inhibit neointimal hyperplasia in an arterial model: a 2% loading dose was used, allowing 10 ug of drug to be released per day from the EVAc polymer (Brauner et al., J Thorac Cardiovasc Surg, 114: 53-63, 1997).

### Delivery Formulations

In selecting a drug delivery system, the following factors must be considered: the solubility of the drug, its molecular weight, the amount of drug needed to be released per day from the polymer, and the desired time and kinetics of drug release to be achieved. Since both Gleevec and rapamycin are very hydrophilic and have molecular weights of less than 1000, they are expected to be rapidly released from a microsphere over a period of hours to days, and therefore may not be helpful in our pig model. To provide for longer durations of release, we have developed three delivery systems that will allow the drugs to be delivered over the course of approximately one month.

Microsphere Loaded Drugs

The expected delivery time for rapamycin or Gleevec from a drug-loaded microsphere is expected to be about 1 to 2 weeks (with some enhanced release initially). This release time is extended when the drug is dispersed in a biodegradable core and this core is enveloped by an outer layer of a non-drug containing biodegradable material, i.e., a double-walled system. This system is expected to work particularly well for Gleevec, because it will allow us to achieve a relatively high % loading amount. The inside core will contain polylactide co gycolide (PLGA) and the outside is composed of polylactide (PLA). Typical dimensions of the microspheres are from about 50 to 200 microns with an outer layer of 5 to 20 microns. Microspheres that are made from biodegradable polymers are placed into the area surrounding the graft at the time of graft placement. A major advantage of these microspheres is that additional microspheres can be injected at later stages. To limit microsphere dispersal from the site of implantation, microspheres can be embedded in an adherent paste such as carboxymethyl cellulose. This will allow the surgeon to deposit the microspheres around the graft, by plastering the vein and graft with a thin layer of paste. It is expected that approximately 200-400 mg of microspheres are embedded in a paste and plastered in a thin layer around the venous anastomotic site. The microsphere paste will extend about 2 cm from the heel of the graft on either side and is placed about 1 cm or so around the graft as well.

Drug Loaded Film and Suture

The therapeutic compounds, alone or in combination, can also be delivered in a nonerodible film that incorporates the drug inside a matrix. The release time for the drug from such a system is expected to be about 3 weeks, with more rapid release initially. Preferably, the polymer is ethyl vinyl acetate (EVAc). EVAc has been widely used for many local delivery applications as well as for perivascular delivery. The film is obtained as detailed below, then wrapped around the venous anastomotic site as well as the junction of the graft with the vein. This is a particularly simple system to make. Based on our initial results, it is likely to provide reasonably linear kinetics over a 2-3 week period, especially if the loading is below 5%. Sutures might be applied around the graft and the vein to hold the film in place.

Alternatively, the drugs are embedded in a polymer suture. The suture is then wound around the venous end of the graft and around the graft at its junction with the vein. This winding of the suture around the graft holds the graft in place. By varying the number of turns of the suture per length of the graft/vein the amount of drug released may be increased or decreased.

Composite System

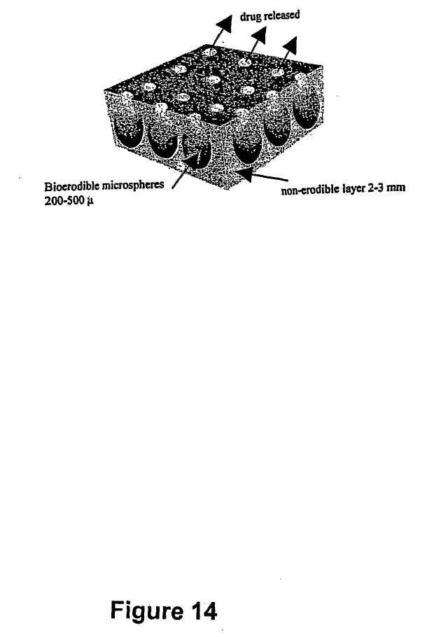

Rapamycin, Gleevec, or another approporiate drug can be delivered from a composite system that combines erodible microspheres embedded within a hydrophobic nonerodible matrix, such as EVAc (see FIG. 14). The major advantages of this system are i) that the microspheres can be loaded to a very high density, for example, up to 50%; ii) that the release can be limited to one side of a film (see schematic below) and can be limited to only those microspheres in contact with the side of the graft or vein; and iii) that the expected drug release, even for small molecular weight hydrophilic drugs, such as Gleevec and rapamycin, is expected to be linear over long periods, such as months or years. The composite system is the most applicable to the human situation. We expect that the thickness of such a composite is about 2-3 mm and that the composite material is wrapped around the graft and the vein.

The composite system will contain polyurethane, EVAc, or other materials known in the art, that are useful to incorporate microspheres in film. In one embodiment, solvent casting is used to embed the microspheres. For this application, a polymer is identified that is soluble in a solvent, in which the microspheres are not soluble (Kohler et al., J Vasc Surg, 30:744-751, 1999).

### Drug Extraction

Gleevec is available in capsules that contain inert substances in addition to the drug. Such capsules are broken, and the drug is extracted in a hydrophilic solvent to separate it from inert materials, and the extracted material is then quantitated. For Gleevec, the inactive ingredients are colloidal silicone, silicone dioxide, crospovidone magnesium stearate, and microcrystalline cellulose. Once the capsule has been broken, the contents are dispersed in an aqueous buffer (pH<5), in which Gleevec is extremely soluble. After lyophilization, purified Gleevec is obtained, and incorporated in an appropriate matrix.

Pure rapamycin is available from Sigma (R0395) or can be extracted capsules as described for Gleevec. The rapamycin extract is then quantified using standard mass spectroscopic methods.

### Polymer Preparation Methods.

Microsphere Preparation

The modified solvent evaporation technique constitutes a nonsolvent bath containing water with 0.5% polyvinyl alcohol solution as a surfactant. The polymer solutions are dissolved in methylene chloride, which is a highly volatile solvent with a boiling point of 40° C., and is miscible with water, the nonsolvent. The agitator speed is set at 500-800 rpm. The agitator controls both the size of droplets formed in the nonsolvent and the rate at which methylene chloride evaporates. At a concentration of 20% weight/volume (w/v), the starting polymer solution of PLLA and PLGA is miscible. As the methylene chloride evaporates, the polymer solution is concentrated and phase separation takes place, resulting in the formation of double walled microspheres. A 400 ml beaker containing 200 ml of deionized water and 50 ml of 2% polyvinyl alcohol solution is used for the fabrication of blank microspheres. A 600 ml beaker containing 400 ml water and 100 ml 2% surfactant is used for the fabrication of protein loaded microspheres.

Microspheres are prepared from a mixture of two polymers—poly(L-lactide) (PLLA) and poly(L-lactide-co-glycolide) 50:50 (PLGA 50:50). The molecular weight of PLGA is adjusted from 20,000 to 100,000 in order to control the release rate. A total of 0.6 g of PLGA and 0.2 g of PLLA are each dissolved in 2 ml of methylene chloride at an overall polymer concentration of 20% weight/volume. The PLGA solution is put on a rotomixer (Fisher Roto-Rack) for 45 minutes to aid solubility in methylene chloride. The 10% w/v PLLA solution is added to the 30% w/v PLGA solution (ratio of 1:3) and injected into the bath using a 10 cc syringe. The bath is allowed to run for 2-3 hrs before the spheres are washed with 500 ml of distilled water and then lyophilized. For encapsulation of drug loaded microspheres, a similar procedure issued. In this case, a total of 0.6 g of PLGA and 0.2 g of PLLA issued. Drug (amount to vary depending on the loadings—1 to 2% for rapamycin and 30 to 50% for Gleevec, as described above) is mixed with 0.1 g of PLGA in 10 ml of methylene chloride. The mixture is bath sonicated for 5 minutes and then the mixture is refrigerated overnight. The next day the methylene chloride is evaporated. 0.5 g of the remaining PLGA is added to 2 ml of methylene chloride and the mixture is put on a rotomixer to dissolve the polymer. The PLGA solution is added to the PLGA-protein vial and bath sonicated for 30 sec. A separate solution of PLLA in methylene chloride is made, 0.2 g in 2 ml of solvent. The two polymeric solutions are then mixed together in a third vial, hand shaken a few times, and then injected into the 600 ml beaker bath consisting of 400 ml of distilled water and 100 ml of 2% polyvinyl alcohol. The bath is stirred for 2-3 hours before the spheres are washed with 500 ml of distilled water and then lyophilized. These microsphere can be used directly, as described above, or can be incorporated into a film.

### EVAc Films and Sutures

EVAc Films

Films are produced by mixing micronized drugs with EVAc, allowing the matrix, which has a thickness of 0.5 to 1.0 mm, to solidify in a petri dish, and cutting rectangular sections of approximately 2 cm×3 cm in size, having a thickness of about 0.5-1.0 mm. Alternatively the mixture of drug and EVAc can be extruded as described below, to form sutures.

EVAc Suture Fabrication

10 ml of poly (ethylene-co-vinyl acetate) (EVAc, 10% w/v in MeCl₂) solution is prepared. Ground powdered drug is added to this solution to achieve loadings of 1 to 2% for rapamycin and 30 to 50% for Gleevec (weigh/weight) depending upon the formulation. Such suspensions are agitated manually, sonicated to disperse the drug, and then cast into a thin film on glass using a scrim set at a height of 200 μm. Upon drying the drug-loaded EVAc film is cut into small pieces. The pieces are fed into a custom extrusion apparatus and heat-extruded through 200-800 μm diameter dies at a temperature of 50-55° C. Based on our previous results, we expect the diameter of the suture to be from 0.5-1.0 mm.

Composite Delivery Systems

Microspheres (made of PLLA and PLGA) are prepared as described above. They are then pressed into a thin flat film is maintained above its glass transition, using an automated hydraulic press with temperature control (Carver, Inc., Wabash, Ind.). This procedure coats the microspheres with EVAc. An additional coat of EVAc is applied using an air brush (Badger Air-Brush Co., Franklin Park, Ill.) on one side of the film. This creates a system that releases drug primarily from one side of a film. Alternatively, the microspheres are prepared and disperses in a polymer that is soluble in solvent that does not dissolve the PLGA. The solvent is then evaporated leaving microsphers embedded in the external polymer. The external polymer used is EVAc or any other non-erodible compatible polymer, such as polyurethane.

### Scanning Electron Microscopy Analysis

Scanning Electron Microscopy (SEM) analysis allows us to determine the degradation of the bioerodible systems over time. Samples are evaluated before and after degradation in vitro, as described below. The purpose of the SEM analysis is to evaluate the external morphology as well as the cross section of each system. Samples for SEM analysis (Hitachi S-2700) are post-fixed at 1% osmium tetroxide (EMS, Fort Washington, Pa.), dehydrated in a series of graded ethanols and placed in hexamethydisilazane (Sigma).

### Fourier-Transform Infrared Spectroscopy (FTIR)

FTIR analysis is used to determine the ratio of PLG to EVAc in the composite delivery devices. FTIR spectra is obtained and analyzed using Perkin-Elmer IR Data Manager software on a model 1725 FTIR spectrophotometer (Perkin-Elmer Corp., Norwalk, Conn.). Microspheres samples are prepared in one of two ways. Some samples are prepared by grinding a 1:99 w/w ratio of microspheres and potassium bromide (KBr, Aldrich Chemical Co., Milwaukee, Wis.) into a fine powder with a mortar and pestle. The mixture is then compressed into a pellet using a Quick Press (Perkin-Elmer Corp., Norwalk, Conn.), or the samples are prepared by film casting. A 1.0% (w/v) polymer/methylene chloride solution is prepared and dispensed onto a NaCl crystal. The solvent is then allowed to evaporate completely before spectra are obtained.

### Considerations for the Combination of Two Drugs

Individual systems for the drug delivery may be combined. For example, rapamycin or another mTOR inhibitor may be delivered in the microspheres and Gleevec may be delivered in the sutures. To deliver the combination, the suture may be wound around the graft while the microspheres are plastered on the graft as a carboxymethyl cellulose paste. Alternatively, microspheres (loaded with each drug at the desired concentration) are embedded in film and delivered to the graft as described above.

### Assessment of Release Kinetics

Preferably, approximately 50% drug release is obtained with roughly linear kinetics over 30 days. The kinetics of release are determined for the various polymeric systems of a 30 and 50% w/w polymer for Gleevec and 1 and 2% w/w polymer for rapamycin. These polymers are placed in 10 ml tissue culture dishes containing standard medium and repeated aliquots are taken over a 4 week period. Following each sampling, fresh medium is added to the tissue culture dish. Aliquots are taken daily, especially if we see an initial burst of release, and then a couple of times each week. These samples are frozen and Gleevec and rapamycin release into the media is quantitated by mass spectroscopy. At the end of four weeks, the matrix is dissolved in an appropriate organic solvent and the remaining drug is measured.

These methods provide the release kinetics of the various drug delivery systems. A summation of the amounts in the sampled aliquots adjusted for volume of the medium in the tissue culture dish plus the drug remaining in the matrix will allow us to assess the release of the drug in the matrix over this 4 week time period.

### Biological Activity

In addition, the biological activity of the drug released from the polymer is assessed using a VSMC migration assay (as described above). The activity of the drug released from the polymer, which is collected at weekly intervals for the duration of the experimental protocol, is compared with the activity of a corresponding amount of drug that has not been embedded in a polymer.

Drug delivery systems are evaluated in 3 pigs to ensure that the systemic level of drugs is acceptable: initially, the drug delivery will not exceed 50 ng/ml for Gleevec and 0.1 ng/ml for rapamycin. Drug serum levels are measured for each of the delivery systems at day 4, 7, and 14 after implantation. Approximately 95% confidence limits for the mean level at each time point are determined. If the upper confidence limit is below the cutoff, we will assume that the systemic level of drug is acceptable and proceed with the experiments outlined below.

If the systemic levels of drugs are lower than 50 ng/ml for Gleevec and 0.1 ng/ml for rapamycin, we can assume—based on in vitro data on effects on VSMCs—that these systemic levels will not effect grafts on the other side of the pig. This will allow us to evaluate two grafts per pig, each graft having different drugs incorporated for local delivery. Thus, we can assess two of the four treatments (Gleevec; rapamycin; combination; or delivery device only) within one animal. If systemic levels are higher than those indicated on any of these days, then the same drug/polymer is tested bilaterally in each of 4 groups of pigs i.e. each pig having two identical drug polymers.

Preferably, the following evaluations are carried out.

Drug Release in Vivo

The amount of drug left in the polymer at the end of the experiment is determined. This involves removing nonerodible polymers from the area of the graft, dissolving these in an appropriate organic solvent, and extracting the drug in water to determine its concentration.

Analysis of Graft with Polymer Device

As described above, the histology of the graft and the venous anastomotic site is evaluated. This evaluation includes detailed cellular phenotyping, cytokine, and intracellular signaling studies. In addition, transmission electron microscopy (TEM) is used to evaluate the morphology of all delivery systems after removal of the device (and prior to placement), most important for the microspheres and the composite system options, both of which contain some biodegradable components. Also, the electron microscopy data will complement data from our cellular phenotyping studies (distinguishing VSMCs from myofibroblasts, for example).

Specimens for TEM are dehydrated in 100% ethanol, osmicated with OSO₄, embedded in LR WHITE embedding media in gelatin capsules, and cured in a 30° C. oven. Sections are cut to a thickness of 95 nm with a diamond knife on a REICHERT-JUNG ULTRACUT E MICROTOME. A PHILIPS EM 410 transmission electron microscope is used to examine sections.

Local (and Systemic) Toxicity

Local necrosis and breakdown of the vascular anastomotic site is evaluated. If toxicity is seen with the drug-loaded polymer side only, the amount of drug added to the polymer is decreased. Of course there is an assessment of whether there is any systemic toxicity from the loaded matrices. This is determined by evaluating whether the animals are eating well, whether they seem alert and responsive. In addition, blood is drawn to assay CBC, hemoglobin and platelet count.

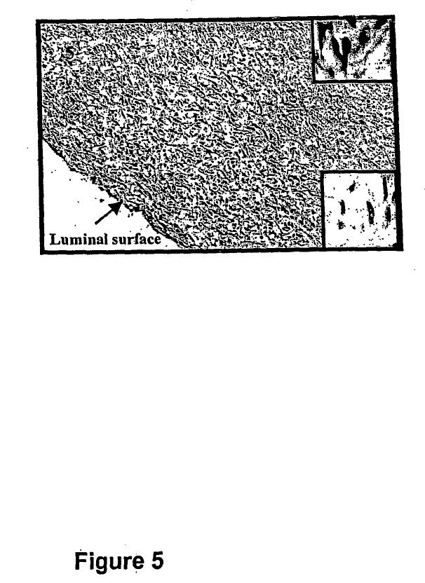

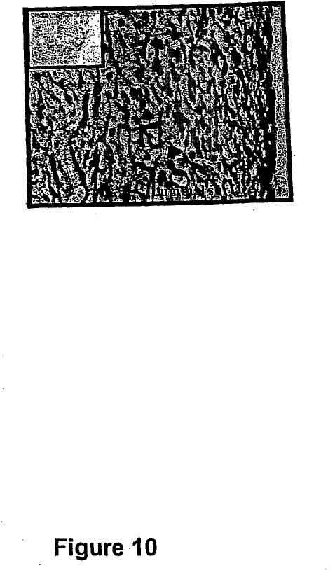

Our methods for ascertaining cytokine and intracellular signaling are carried out as described above. For rapamycin we determine whether rapamycin has hit its target (mTOR) using a rapamycin sensitive phospho p70 S6 kinase antibody as shown in FIGS. 5 and 10. We expect to see negative staining results, demonstrating that rapamycin has blocked mTOR activation. If we observe positive staining results, higher amounts of rapamycin are used to (for example 10 fold) load the polymer with drug.

Data Analysis

As described above, a mixed model analysis of variance or a generalized estimating equation is used to analyse this serial data. These approaches will correctly account for the relationship between measurements over time, and between treatments when tested in the same animal, while allowing measurements from all animals to be incorporated in the analysis.

### Optimal Locally Delivered Drug Formulation

Perindopril or vehicle is administered to each animal having one graft with drug (Gleevec, rapamycin, or Gleevec and rapamycin) and one graft with the delivery system only. In particular, the comparison of local delivery system+perindopril vs. delivery system alone will need to be a between group comparison. A preliminary analysis is carried out after completing six sets of animals to determine whether there is any evidence that drugs administered in combination with perindopril are substantially better than the drug treatment in the absence of perindopril. Data analysis follows the same strategy and methods described above, allowing us to properly account for comparisons within and between animals.

### Compounds Useful for Local Administration in the AV Graft Model

In addition to rapamycin, Gleevec, and perindopril, drugs that block receptors or intracellular pathways that are known to deliver pro-migratory and/or pro-mitogenic signals (i.e., drugs that block receptors other than PDGF and Ang-II receptors) on VSMCs are also useful in the methods of the invention.

ET-1 Receptor Blockers

For example, if ET-1 expression is observed in the neointimal lesion, as has been noted by Weiss et al., Am J Kidney Dis, 37:970-980., 2001, than ET-1 receptor blockers can be administered perivascularly. Alternatively, intracellular inhibitors, other than rapamycin, such as inhibitors of ERK-1/2 or of PI3K may be administered. PI3K activates Akt as well as other downstream effectors. Such compounds are particularly useful when phosphorylation of such pathways is observed in the presence of Gleevec, rapamycin, and/or perindopril. While such inhibitors might have adverse side-effects if administered systemically, when delivered locally using the methods of the invention, lower effective doses could be administered, thus decreasing adverse side-effects and systemic toxicity.

Suramin

Suramin is one preferred cytokine inhibitor for local administration in the AV graft model. Suramin is a polysulphonated naphthylmine derivative of urea that has been widely used for African trypanosomiasis and onchocercuiasis. Suramin is also effective in the treatment of various neoplasms Small et al., J Clin Oncol, 20:3369-3375, 2002, Ryan et al. Cancer Chemother Pharmacol, 50:1-5., 2002). Recently, suramin was shown to inhibit intimal thickening after balloon angioplasty in a rabbit model when administered systemically Asada et al., Cardiovasc Res, 28:1166-1169, 1994; Urasawa et al., Jpn Heart J, 42:221-233, 2001). Suramin was also effective in inhibiting neointimal hyperplasia in mouse vein grafts when administered locally. Accumulating evidence suggests that Suramin inhibits VSMC proliferation and migration by abolishing PDGF-activated MAP kinase activity and inhibiting the phosphorylation of the PDGF receptor; by blocking Ang-II induced mitogenesis in VSMCs; and by interfering with ligand binding of numerous growth factors (acidic and basic fibroblast growth factors, IGF-I, IL-1 and TGF-β), inhibiting fibronectin and elastin deposition, and increasing the expression of elastin binding protein which might help to anchor the smooth muscle cell to extracellular matrix.

Suramin administration is indicated when expression of bFGF, or TGF-β, or IGF-I is present in the neointimal lesion when the pigs are treated with rapamycin, Gleevec, or perindopril. Given that Suramin's toxicity profile may preclude its chronic systemic use in end stage renal disease (ESRD) patients for the treatment of AV graft stenosis, suramin is administered locally according to the methods of the invention for the treatment of neoinitmal hyperplasia.

### Transcriptional Profiling Analysis on Neointimal Lesions

Initially, AV grafts in pigs are evaluated at different time points (e.g., weekly after graft placement) in the absence of any medication other than aspirin. This allows us to evaluate temporal cytokine expression in the neointimal lesion. Subsequent to this evaluation, pigs are treated with the drugs described herein, and temporal cytokine expression are again monitored. If the reduction of neointimal hyperplasia is absolute then no RNA is obtained from the lesion. If some neointimal hyperplasia tissue remains, cytokine expression in the remaining lesion is analysed. This provides an unbiased view of what compensatory or redundant cytokines occur in the lesion after effective PDGF blockade. A similar analysis is done on animals treated with perindopril and/or with rapamycin. Such evaluations are carried out on porcine microarrays (Moody et al., BMC Genomics, 3: 27, 2002), or on cDNA arrays from mouse or humans, since the pig RNA is expected to cross-hybridize with such cDNA arrays.

### Compound Screening Assays

As discussed above, our experimental results demonstrated that N-phenyl-2-pyrimidine compounds (e.g., imatinib mesylate) and mTOR inhibitors (e.g., rapamycin) are useful to treat graft failure caused by neointimal hyperplasia. Based on this discovery we have also developed a screening procedure for identifying combinations of therapeutic compounds that can be used to prevent or treat graft failure. In general, the method involves screening any number of compounds for therapeutically-active agents by employing the vascular smooth muscle cell migration assays described herein. Accordingly, the methods of the invention simplify the evaluation, identification, and development of active agents, such as drugs, for the treatment of graft failure caused by neointimal hyperplasia. Moreover, identified therapeutic combinations can be evaluated in vivo using the animal models described herein.

In general, the chemical screening methods of the invention provide a straightforward means for selecting natural product extracts or compounds of interest from a large population which are further evaluated and condensed to a few active and selective materials. Constituents of this pool are then purified and evaluated in the methods of the invention to determine their effect on graft failure or neointimal hyperplasia.

### Test Extracts and Compounds

In general, compounds to be used in combination with N-phenyl-2-pyrimidine compounds for the treatment of neointimal hyperplasia are identified from large libraries of both natural product or synthetic (or semi-synthetic) extracts or chemical libraries according to methods known in the art. The screening method of the present invention is appropriate and useful for testing compounds from a variety of sources for possible effects on vascular smooth muscle cell migration. The initial screens may be performed using a diverse library of compounds, but the method is suitable for a variety of other compounds and compound libraries. Such compound libraries can be combinatorial libraries, natural product libraries, or other small molecule libraries. In addition, compounds from commercial sources can be tested, as well as commercially available analogs of identified inhibitors.

For example, those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to have an effect on vascular smooth muscle cell migration, further fractionation of the positive lead extract is necessary to isolate chemical constituents responsible for the observed effect. Thus, the goal of the extraction, fractionation, and purification process is the careful characterization and identification of a chemical entity within the crude extract having an effect on vascular smooth muscle cell migration. Methods of fractionation and purification of such heterogenous extracts are known in the art. If desired, compounds shown to be useful agents for the treatment of neointimal hyperplasia are chemically modified according to methods known in the art.

Since many of the compounds in libraries such as combinatorial and natural products libraries, as well as in natural products preparations, are not characterized, the screening methods of this invention provide novel compounds which are active as inhibitors or inducers in the particular screens, in addition to identifying known compounds which are active in the screens. Therefore, this invention includes such novel compounds, as well as the use of both novel and known compounds in pharmaceutical compositions, therapeutic combinations, and methods of treating patients having graft failure.

### Therapeutic Uses

The invention features methods of treating graft failure resulting from neointimal hyperplasia by administering N-phenyl-2-pylimidine compounds, such as imatinib mesylate, and/or mTOR inhibitors, such as rapamycin. The preferred types of grafts are those used to treat PVD or to establish vascular access in hemodialysis. A compound may be administered alone or in combination with another therapeutic compound, such as those identified according to the methods of the invention, in a pharmaceutically acceptable diluent, carrier, or excipient, in unit dosage form. Administration may be parenteral, intravenous, subcutaneous, oral, or local at the site of the graft.

The composition can be in the form of a pill, tablet, capsule, liquid, or sustained release tablet for oral administration; or a liquid for intravenous, subcutaneous or parenteral administration; or a polymer or other sustained release vehicle for local administration.

Methods well known in the art for making formulations are found, for example, in “Remington: The Science and Practice of Pharmacy” (supra). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Nanoparticulate formulations (e.g., biodegradable nanoparticles, solid lipid nanoparticles, liposomes) may be used to control the biodistribution of the compounds. Other potentially useful parenteral delivery systems include ethylene-vinyl acetate copolymer particles, osmotic pumps, implantable infusion systems, and liposomes. The concentration of the compound in the formulation will vary depending upon a number of factors, including the dosage of the drug to be administered, and the route of administration.

The compound may be optionally administered as a pharmaceutically acceptable salt, such as a non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as talnic acid, carboxymethyl cellulose, or the like; and inorganic acid such as hydrochloric acid, hydrobromic acid, sulfuric acid phosphoric acid, or the like. Metal complexes include zinc, iron, and the like.

### Therapeutic Formulations

Formulations for oral use include tablets containing the active ingredient(s) in a mixture with non-toxic pharmaceutically acceptable excipients. These excipients may be, for example, inert diluents or fillers (e.g., sucrose and sorbitol), lubricating agents, glidants, and antiadhesives (e.g., magnesium stearate, zinc stearate, stearic acid, silicas, hydrogenated vegetable oils, or talc).

Formulations for oral use may also be provided as chewable tablets, or as hard gelatin capsules wherein the active ingredient is mixed with an inert solid diluent, or as soft gelatin capsules wherein the active ingredient is mixed with water or an oil medium.

For oral use of imatinib mesylate preferred dosages range from 50 mg to 5000 mg per day, more preferably 100 mg to 800 mg per day, and most preferably 100 mg to 400 mg per day given in one daily dose, two daily doses, or up to four daily doses.

Systemic administration can also be achieved using the intravenous access necessary for the hemodialysis treatments. This method of systemic administration can circumvent the need for patient compliance and can provide an alternative mechanism when systemic administration is desired and compounds are not available for oral administration.

For local administering, the compound can be directly injected at the graft or venous anastamotic site. In addition, a polymer, which is applied at the site of the graft or the venous anastamotic site, can be used to allow for controlled release of the compound at the graft site. This controlled release form of imatinib mesylate fills the nodal spaces of the PTFE membrane wall and/or coats the inner and/or outer surfaces of the membrane. Examples of such polymers include Gelfoam (Pharmacia & Upjohn Co.), which has been used by surgeons for many years and its application here will be appreciated by those skilled in the art. Another example of a polymer that can be used for local, controlled release of the compound is ethylene-vinyl-acetate (EVA; DuPont Chemical Co.). The incorporation of compounds into EVA for sustained release delivery is also well known in the art (see for example Brauner et al., J. Thorac. Cardiovasc. Surg. 114:53-63, 1997; Edelman et al., Proc. Natl. Acad. Sci. USA 87:3773-3777, 1990; Kusaka et al., Biochem. Biophys. Res. Commun. 174: 1070-1076, 1991; Signore et al., J. Vasc. Interv. Radiol., 12: 79-88, 2001) and standard procedures are used for this method of treatment. In general, percent composition of imatinib mesylate will range from 0.05% to 10% weight for weight of imatinib mesylate to polymeric matrix. One example of these procedures is described in Example 1.

Local administration of the compound can also be achieved through stents or other types of mechanical devices used to support the vessel such as catheters. The stents or catheters, which contain repositories for therapeutic compounds, can be inserted directly into the vessel and the therapeutic compound is released from the repositories as blood flows through the vessel. Methods for coating stents with various pharmaceutical compounds are well-known in the art and examples can be found in U.S. Pat. Nos. 6,153,252; 6,258,121; and 5,824,048 incorporated herein by reference. For each of the therapeutic compounds listed in the present application, the amount of therapeutic agent used is dependent upon the particular drugs employed. Typically, the amount of drug represents about 0.001% to about 70%, more typically about 0.001% to about 60%, and most typically 0.001% to about 45% by weight of the coating.

Preferably, the therapeutic agent is delivered via a drug delivery system described herein (e.g., microsphere, drug loaded film, suture, composite system, or microspheres in film).

The timing of administering the compound will depend on various clinical factors including the overall health of the patient, the diagnosis of graft failure, and the rates of access blood flow. For hemodialysis, one dosage is given at the time of each hemodialysis treatment, and can be continued for a period of time ranging from 1 to 180 days, more preferably 7 to 120 days, and most preferably 7 to 90 days. For PVD by-pass grafts, one dosage can be given at the time of graft placement, and can be continued for a period of time ranging from 1 to 180 days, more preferably 7 to 120 days, and most preferably 7 to 90 days. Repeated applications of the local administration can be required and will include repeated injections, applications of the compound, or replacement of the graft. Repeated application of the systemic administration either by oral administration or intravenous administration can also be required as a maintenance therapy.

### Therapeutic Combinations

As described above, the cellular signaling pathways that initiate and sustain neointimal hyperplasia resulting in graft stenosis and occlusion are numerous and varied. The upstream stimulants can include mitogens and cytokines including platelets, neutrophils or macrophages. Classic proliferation signaling pathways and cell migration signaling pathways, as well as angiogenic signaling pathways, and immunologic response pathways are also upregulated. In addition, the cellular processes leading up to neointimal hyperplasia and graft failure include increased extracellular matrix deposition, cell migration, and cell adhesion. The cellular proteins that regulate each of these aspects of the neointimal hyperplastic lesion are diverse. Therefore, it is advantageous to target several of these pathways when designing therapeutic approaches to combat neointimal hyperplasia and graft failure. The use of additional compounds will likely lead to greater therapeutic success and a concomitant reduction in the toxicity of the compounds and the dosages required.

Accordingly, the present invention provides for a pharmaceutical composition comprising an N-phenyl-2-pyrimidine derivative capable of inhibiting PDGFR biological activity and at least one additional compound selected from the following: (i) an angiogenesis inhibitor, (ii) an anti-proliferative compound, (iii) an immunosuppressive compound, (iv) an anti-migratory compound, (v) an anti-platelet agent, and (vi) an anti-fibrotic compound.

Angiogenesis inhibitors include but are not limited to vascular endothelial growth factor (VEGF) inhibitors such as antibodies against VEGF-A, antibodies against one of the VEGF receptors, and small molecule compounds that inhibit the tyrosine kinase activity of one of the VEGF receptors. Additional examples of angiogenesis inhibitors include endostatin, angiostatin, restin, tumstatin as well as other small molecule inhibitors such as TNP-470, two methoxyestradiol, and thalidomide.

The dosage of the angiogenesis inhibitor will depend on other clinical factors such as weight and condition of the human or animal and the route of administration of the compound. For treating humans or animals, between approximately 0.5 mg/kg to 500 mg/kg body weight of the angiogenesis inhibitor can be administered. A more preferable range is 1 mg/kg to 100 mg/kg body weight with the most preferable range being from 2 mg/kg to 50 mg/kg body weight. Depending upon the half-life of the angiogenesis inhibitor in the particular animal or human, the angiogenesis inhibitor can be administered between several times per day to once a week. The methods of the present invention provide for single as well as multiple administrations, given either simultaneously or over an extended period of time.

Anti-proliferative compounds include any compound, which can reduce or inhibit the proliferation of vascular smooth muscle cells or endothelial cells. Examples of mitogens known to induce proliferation of vascular smooth muscle cells include PDGF, bFGF, endothelin-1, angiotensin II, EGF, IGF-1, vasopressin, thrombin, serotonin, and numerous lipid mediators. Examples of anti-proliferative compounds include any compound that can inhibit the mitogenic effects of any of these or any other proliferation-inducing proteins. Preferred examples of specific compounds include but are not limited to rapamycin, bFGF inhibitors, paclitaxel (taxol), 17 beta-estradiol, troglitazone, ACE inhibitors, statins, and suramins.

The dosage of the anti-proliferative compound depends on clinical factors such as weight and condition of the human or animal and the route of delivery of the compound. In general, for treating humans or animals, between approximately 0.01 mg/kg to 500 mg/kg body weight of the anti-proliferative compound can be administered. A more preferable range is 0.1 mg/kg to 50 mg/kg body weight with the most preferable range being from 1 mg/kg to 25 mg/kg body weight. Depending upon the half-life of the anti-proliferative compound in the particular animal or human, the compound can be administered between several times per day to once a week or once every other week. The methods of the present invention provide for single as well as multiple administrations, given either simultaneously or over an extended period of time.

For rapamycin, dosages are provided to produce a blood level of rapamycin that ranges from 0.001 μg/ml to 10 μg/ml with a preferred range of 0.005 μg/ml to 0.1 μg/ml. Taxol is administered intravenously at weekly dosages ranging from approximately 0.5 mg/kg to 5 mg/kg body weight. A more preferable range is 1 mg/kg body weight to 5 mg/kg body weight with the most preferable range being from 1 mg/kg to 2.5 mg/kg body weight. Troglitazone is given orally or intravenously at daily dosages ranging from approximately 0.5 mg/kg to 25 mg/kg body weight. A more preferable range is 1 mg/kg body weight to 20 mg/kg body weight with the most preferable range being from 1 mg/kg to 10 mg/kg body weight.

Statin is the common name for a class of drugs formally known as 3-hydroxy-3-methylglutaryl coenzyme A (HMG CoA) reductase inhibitors. These drugs lower levels of low-density lipoprotein cholesterol. Smooth muscle cell proliferation is a feature of atherogenesis and therefore, drugs affecting this metabolic pathway, such as statins, may reduce smooth muscle cell proliferation. Statins now marketed in the United States include atorvastatin (Lipitor), fluvastatin (Lescol), lovastatin (Mevacor), pravastatin (Pravachol), simvastatin (Zocor), cerivastatin (Baycol, removed from market in 8/01), and a number of other formulations. Dosages for statins may be obtained for each formulation from the pharmaceutical manufacturer. Recommended dosages range from 0.20 to 100 mg daily depending on the particular formulation being used. For example, the recommended daily dosage for fluvastatin is 20 to 40 mg.

Angiotensin-converting enzyme (ACE) inhibitors block the effects of angiotensin II, which is a potent vasoconstrictor. In addition to its vasoconstricting properties, angiotensin also stimulates vascular smooth muscle cell proliferation. ACE inhibitors can be effective at preventing restenosis both through the prevention of vascular recoil and remodeling and the prevention of inflammation and cell proliferation. There are several ACE inhibitors currently marketed in the United States. Examples include perindopril (Aceon), quinapril, captopril (Capoten), Lisinopril (Prinivil, Zestlil), enalapril (Vasotec), fosinopril (Monopril), benazepril (Lotensin), cilazapril, and ramipril (Altace). Dosages for ACE inhibitors may be obtained for each formulation from the pharmaceutical manufacturer. Recommended daily dosages range from 0.1 to 500 mg depending on the particular formulation being used. For example, the preferred dosage for perindopril is 2 mg to 20 mg per day, while the preferred dosage for Capoten is 25 to 150 mg/b.i.d. or t.i.d. with a maximum dosage of 450 mg per day.

Suramin is a polyanionic compound that, after decades of use as an anti-parasitic drug, was recognized for its ability to block autocrine and paracrine growth factors required for the proliferation of smooth muscle cells. Suramin has recently been used in animal studies for the treatment of neoplasms. Suramin is marketed under several brand names including Antrypol and Surmontil. Dosages for suramin may be obtained for each formulation from the pharmaceutical manufacturer. Recommended daily dosages range from 50 to 200 mg depending on the particular formulation being used.

Immunosuppressive compounds include any compounds which can suppress the natural immune response of an animal. Preferred compounds will suppress the activity of monocytes and/or macrophages, which play a central role in the chronic inflammatory response. Preferably, an immunosuppressive compound can inhibit monocyte or macrophage activity in any one of four ways: 1) by decreasing the production or increasing the removal of monocytes, 2) by preventing differentiation of monocytes into macrophages, 3) by preventing monocytes from migrating into the region of hyperplasia, and 4) by blocking the activation of macrophages. Non-limiting examples of preferred immunosuppressive compounds include antibodies or compounds that block the production or functioning of mac-1, any compound that blocks MCP-1 production or function or both, antioxidants such as alpha-tocopherol, and compounds that block P-selectin, PSGL-1, VLA-4 or VCAM-1. Steroids are another example of a class of compounds that inhibit macrophage activity. Non-limiting examples of steroids include prednisone and methylprednisolone. Dosages for each compound can be obtained from the manufacturer and will vary depending on the weight and condition of the patient and the route of administration of the compound. Another example of an immunosuppressive compound is FTY720, which is currently under development by Novartis. Dosages for FTY720 are determined based on the information gained from Phase II and Phase III clinical trials. Current dosages based on preliminary studies range from 2 mg/day to 5 mg/kg per day.

Anti-migratory compounds include any compound that reduces or prevents the movement or migration of smooth muscle cells. Specific, non-limiting examples include serotonin (5-HT2) receptor antagonists, endothelin-1 receptor antagonists, and vasopressin receptor antagonists. Endothelin-1 (ET-1) is a potent vasoconstrictor secreted by endothelial and smooth muscle cells. It has two receptors, ET_{A} located on vascular smooth muscle cells and ET_{B} located on endothelial and vascular smooth muscle cells. Endothelin-1 stimulates vascular smooth muscle cell proliferation and migration alone and in combination with other cytokines. It also stimulates extracellular matrix synthesis. There is some evidence to suggest that both ET-1 and angiotensin II can act synergistically on vascular smooth muscle cells. There is also evidence based on animal studies that ET-1 receptor antagonists and ET-converting enzyme inhibitors are useful in the prevention of neointimal hyperplasia. These data suggest that endothelin-1 receptor antagonists might be useful in the prevention of migration of smooth muscle cells.

Serotonin is secreted mainly by platelets and acts on blood vessels via the 5-HT₁ and 5-HT₂ receptors. Over the past decade, some researchers have suggested that serotonin may be involved in vascular smooth muscle cell proliferation and migration. The 5-HT₂ receptor antagonists Ketanserin and Anplag were shown to be effective in the prevention of neointimal hyperplasia in rabbit carotid artery balloon angioplasty and vein graft intimal hyperplasia models suggesting that they may be useful in the prevention of proliferation and migration of smooth muscle cells.

Specific examples of anti-migratory compounds include cyproheptadine, bosentan, Ketanserin, Anplag and YM087. Dosages for each compound can be obtained from the manufacturer and will vary depending on the weight and condition of the patient and the route of administration of the compound.

Anti-platelet agents can include any cyclooxygenase inhibitor (e.g., aspirin), ADP inhibitor (e.g., ticlopidine), phophodiesterase III inhibitor (e.g., cilostazol and dipyridamole), or glycoprotein IIB/IIIA inhibitor (e.g., abciximab). Dosages for each compound can be obtained from the manufacturer and will vary depending on the weight and condition of the patient and the route of administration of the compound.

Anti-fibrotic agents include antagonists of transforming growth factor beta (TGFβ) or connective tissue growth factor (CTGF). The dosage of the anti-fibrotic agent will depend on other clinical factors such as weight and condition of the human or animal and the route of administration of the compound. For treating humans or animals, between approximately 0.1 mg/kg to 500 mg/kg body weight of the anti-fibrotic agent can be administered. A more preferable range is 1 mg/kg to 50 mg/kg body weight with the most preferable range being from 1 mg/kg to 25 mg/kg body weight. Depending upon the half-life of the anti-fibrotic agent in the particular animal or human, the anti-fibrotic agent can be administered between several times per day to once a week. The methods of the present invention provide for single as well as multiple administrations, given either simultaneously or over an extended period of time.

For each of the compounds listed, the specific compound can be given with just the PDGFR inhibitor or in combination with any other additional compound listed. In one example, a PDGFR inhibitor can be administered in combination with rapamycin and, if desired, a steroid such as prednisone can also be administered.

The compounds are administered individually or simultaneously by any means determined to be most effective and least toxic for each individual compound. For example each compound can be given orally, locally, systemically, or in any combination of oral, systemic and local administration. For each of the compounds listed, local administration at the site of the graft or AV fistula can also be used. If local administration is desired, the methods described herein are be used to determine the optimal drug compound composition in the polymeric matrix. In general, percent composition of the compound will range from 0.05% to 10% weight for weight of compound to polymeric matrix.

Therapeutic Compounds in Combination with Radiation Therapy

The invention also provides for the use of a therapeutically effective amount of a compound capable of inhibiting PDGFR biological activity in combination with radiation therapy. It will be appreciated that there will be many ways known in the art to determine the dosage and duration of treatment. For radiation therapy, gamma rays or X-rays are used in an amount sufficient to induce enough damage to a cell so as to limit its ability to function normally or to destroy the cell altogether. Typical dosages range from 10 to 200 units (Grays) per day.

It should be noted that although each of the compounds is listed under a specific category of compounds, these categories are not meant to be limiting in scope. Many of the compounds possess more than one activity and can therefore be included under more than one category. For example, serotonin receptor antagonists can have both anti-proliferative and anti-migratory activity.

### Methods for Determining Efficacy

Patients treated with the therapeutic compounds or combinations of the invention are typically followed by a physician to track the success of the treatment. Indications of treatment failure include patient complaints of symptoms returning, failure of standard stress tests, and return of disease indicators. In addition, quantitative angiography is often used to determine lumen diameter of the treated vessel. An increase of 25% or more, preferably 50% or more, and most preferably 75% or more in lumen diameter post-treatment as compared to pre-treatment is indicative of therapeutic efficacy. The diameter of the treated vessel can also be compared to a reference distal and proximal segment to determine therapeutic efficacy. Additional methods for measuring therapeutic efficacy include magnetic resonance angiography and intravascular ultrasound (IVUS), which allows for quantitation of neointimal formation, luminal diameter, plaque area and volume. The patient is monitored both in the short-term (up to six months after initial treatment) and the long-term (six months or more after the initial treatment) to determine the complete efficacy of the treatment using the compounds of the present invention.

Dosages and toxicity for compounds can be measured in vivo using the animal models described below. In addition, an in vitro system using cultures of smooth muscle cells can be used to determine effective dosages of each compound as well as compound stability. For example, a specific number of cells can be plated in culture, stimulated with known mitogens such as growth factors or angiotensin II, and treated with increasing concentrations of each drug compound. Each day, the number of cells in the culture can be counted and proliferation measured (see Powell et al., supra). A positive result is considered to be a decrease in proliferation rate of at least 10%, preferably 25% and more preferably 50% or more as compared to cells treated with mitogen alone.

### Animal Studies

The method of treatment provided in the present invention also includes administering a therapeutic amount of an N-phenyl-2-pyrimidine derivative or an mTOR inhibitor to a warm-blooded animal suffering from neointimal hyperplasia or vascular stenosis, in a dose sufficient to prevent or ameliorate neointimal hyperplasia and vascular stenosis. Such animals may also be treated with a combination of the N-phenyl-2-pyrimidine derivative and one of the following: (i) an angiogenesis inhibitor, (ii) an anti-proliferative compound (iii) an immunosuppressive compound, (iv) an anti-migratory compound, (v) an anti-platelet agent, and (vi) an anti-fibrotic compound.

Warm-blooded animals, as used herein, include but are not limited to mice, dogs, pigs, baboons, and monkeys. Also included are any animal models of neointimal hyperplasia or vascular stenosis which can be used to study dosages and efficacy of treatment (see for example Sukhatme V. P., Kidney Intl. 49:1161-1174, 1996; Kelly et al., J. Am. Soc. Nephrol, 12:A1496, 2001; Kelly et al., Current Surgery, 57:18, 2000). The working examples described above feature exemplary uses of animal models for the study of treatment of venous neointimal hyperplasia or venous stenosis.

### Other Embodiments

From the foregoing description, it is apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A method for the prevention or treatment of graft failure resulting from neointimal hyperplasia comprising administering to a patient an effective amount of an mTOR inhibitor, wherein said administering is in a dose sufficient to prevent or treat said graft failure.

2. The method of claim 1, wherein said graft is used for vascular access in hemodialysis.

3. The method of claim 2, wherein said vascular access for hemodialysis is associated with the use of a polytetrafluoroethylene (PTFE) graft.

4. The method of claim 2, wherein said vascular access is associated with the use of an arteriovenous fistula.

5. The method of claim 1, wherein said graft is used to treat peripheral vascular disease.

6. The method of claim 5, wherein said graft is comprised of a synthetic material.

7. The method of claim 6, wherein said synthetic material is Dacron.

8. The method of claim 5, wherein said graft comprises a portion of vein from the patient who will receive the graft.

9. The method of claim 1, wherein said graft failure is characterized by the migration of smooth muscle cells into the intima.

10. The method of claim 1, wherein said graft failure is characterized by the proliferation of vascular smooth muscle cells.

11. The method of claim 1, wherein said graft failure is characterized by the deposition of extracellular matrix.

12. The method of claim 1, wherein said graft failure is the result of vascular stenosis or thrombosis.

13. The method of claim 1, wherein said compound is given in combination with a pharmaceutically acceptable carrier.

14. The method of claim 1, wherein said dosage is sufficient to prevent or ameliorate vascular stenosis or thrombosis.

15. The method of claim 1, wherein said dosage is sufficient to prevent or ameliorate neointimal hyperplasia.

16. The method of claim 1, further comprising administering to a patient at least one additional compound selected from the group consisting of:
(a) an angiogenesis inhibitor,
(b) an anti-proliferative compound,
(c) an immunosuppressive compound,
(d) an anti-migratory compound,
(e) an anti-platelet compound, and
(f) an anti-fibrotic compound.

17. The method of claim 16, wherein said additional compound is an angiogenesis inhibitor selected from the group consisting of an antibody that binds VEGF-A, an antibody that binds a VEGF receptor and blocks VegF binding, avastin, endostatin, angiostatin, restin, tumstatin, TNP-470, 2-methoxyestradiol, thalidomide, a peptide fragment of an anti-angiogenic protein, canstatin, arrestin, a VEGF kinase inhibitor, CPTK787, SFH-1, an anti-angiogenic protein, thrombospondin-1, platelet factor-4, interferon-α, an agent that blocks TIE-1 or TIE-2 signaling, or PIH12 signaling, an agent that blocks an extracellular vascular endothelial (VE) cadherin domain, an antibody that binds to an extracellular VE-cadherin domain, tetracycline, penicillamine, vinblastine, cytoxan, edelfosine, tegafur or uracil, curcumin, green tea, genistein, resveratrol, N-acetyl cysteine, captopril, a cox-2 inhibitor, celecoxib, and rofecoxib.

18. The method of claim 17, wherein said additional compound is an anti-proliferative compound selected from the group consisting of taxol, troglitazone, an antibody that binds bFGF, an antibody that binds bFGF-saporin, a statin, an ACE inhibitor, suramin, 17 beta-estradiol, atorvastatin, fluvastatin, lovastatin, pravastatin, simvastatin, cerivastatin, perindopril, quinapril, captopril, lisinopril, enalapril, fosinopril, cilazapril, ramipril, and a kinase inhibitor.

19. The method of claim 17, wherein said additional compound is an immunosuppressive compound selected from the group consisting of prednisone, FTY720, methylprednisolone, α-tocopherol, azathioprine, chorambucil, cyclophosphamide, an antibody that binds to an IL-2 receptor or to CTLA4, methotrexate, mycophenolate mofetil, cyclosporine, an agent that interferes with macrophage function, an agent that inhibits P-selectin PSGL-1, VLA-4, VCAM-1 or that blocks that Mac-1 biological function, and FTY720.

20. The method of claim 17, wherein said additional compound is an anti-migratory compound selected from the group consisting of cyproheptadine, methysergide, bosentan, YM087, ketanserin, and anplag.

21. The method of claim 17, wherein said additional compound is an anti-platelet compound selected from the group consisting of ticlopidine, cilostazol, dipyridamole, abciximab, clopidogrel, a glycoprotein iib/iiia inhibitor, eptifibatide, tirofiban, and a phosphodiesterase III inhibitor.

22. The method of claim 17, wherein said additional compound is an anti-fibrotic compound selected from the group consisting of an agent that blocks TGF-β signaling or inhibits activation of plasminogen activator inhibitor-I promoter activity, an antibody that binds to TGF-β or to a TGF-β receptor, an antibody that binds to TGF-β receptor I, II, or III, a kinase inhibitor, an agent that blocks connective tissue growth factor (CTGF) signaling, an agent that inhibits prolyl hydroxylase, an agent that inhibits procollagen C-proteinase, pirfenidone, silymarin, pentoxifylline, colchicine, embrel, remicade, an agent that antagonizes TGF-β, an agent that antagonizes CTGF, and an agent that inhibits vascular endothelial growth factor VEGF.

23. The method of claim 1, wherein said mTOR inhibitor is administered to said patient orally.

24. The method of claim 1, wherein said mTOR inhibitor is administered to said patient locally.

25. The method of claim 1, wherein said mTOR inhibitor is rapamycin or a rapamycin analog.
